(19) United States
(12) Patent Application Publication
(10) Pub. No.: US 2003/0135172 A1
(43) Pub. Date: Jul. 17, 2003

(54) Absorbent article

(76) Inventors: Darryl L. Whitmore, Chesapeake, VA (US); Friedrich Engelhardt, Frankfurt/Main (DE)

Correspondence Address: MARSHALL, GERSTEIN & BORUN, 6300 SEARS TOWER, 233 SOUTH WACKER, CHICAGO, IL, 60606-6357, US

(21) Appl. No.: 10/300,082

(22) Filed: Nov. 20, 2002

Related U.S. Application Data
(60) Provisional application No. 60/341,254, filed on Dec. 20, 2001.

(51) Int. Cl. A61F 13/15

(52) U.S. Cl. 604/359; 604/368

(57) ABSTRACT

A storage layer obtainable by a process including (a) forming a sprayable blend containing one or more superabsorbent forming monomers; superabsorbent polymer particles; water; and one or more initiators; (b) applying the sprayable blend to a fibrous web; and (c) subjecting the sprayed fibrous web to conditions under which the superabsorbent forming monomer polymerizes. The storage layer is used in absorbent articles to store aqueous fluids.

### CROSS REFERENCE TO RELATED APPLICATIONS

This application claims the benefit of U.S. provisional patent application Serial No. 60/341,254, filed Dec. 20, 2001; and is a continuing application of PCT patent application PCT/EP02/05533, designating the U.S., which claims the benefit of U.S. provisional patent application Serial No. 60/292,523, filed May 23, 2001; and is a continuing application of PCT patent application PCT/EP02/05534, designating the U.S., which claims the benefit of U.S. provisional patent application Serial No. 60/292,511, filed May 23, 2001.

### FIELD OF THE INVENTION

The present invention relates to a storage layer for use in an absorbent article, such as in hygiene products.

### BACKGROUND OF THE INVENTION

Superabsorbent polymers used in personal care products are designed to absorb body fluids, such as urine, blood, menses, and the like. Generally, body fluids contain malodorous components. Such malodors develop after contact with air and bacteria for prolonged periods. Furthermore, absorbed fluid exudates are converted to ammonia by urease produced by skin-florea, which can lead to skin irritation. Odor control materials already have been used in superabsorbent polymers. Odor control materials known in the art include zeolites, silica, activated carbon, chelants, antimicrobial agents, perfuming ingredients, masking agents, enzymes, peroxides, hydrogen carbonates, vegetable extracts, etherical oils, boron derivatives, poly-alpha-amino acids, imides, polyimides, pvp-iodine, chitosan, polyglycoside, and cyclophanes, see, for example, U.S. Pat. No. 4,795,482; EP-B-0,297,543; EP-A-0 341 951; U.S. Pat. Nos. 4,990,338; and 5,035,892.

U.S. Pat. No. 5,037,412 describes the use of an odor control material which absorbs odor compounds of each level of pH. Materials for absorption of acidic odors are inorganic carbonates, bicarbonates, phosphates, biphosphates, sulfates, bisulfates, or mixtures thereof with pH of higher than 7. These inorganic compounds are present in the odor control material from 40-65% of the whole mixture. Absorption of odors of pH of higher than 7 is achieved by materials of pH of lower than 7, e.g., ascorbic acid, stearic acid, citric acid, maleic acid, and polyacrylic acid. These compounds are added from 30-45% to the mixture. Neutral odors are absorbed by activated carbon, silica, polystyrene derivatives, zeolites, molecular sieve, and starch with contents up to 10% of the whole mixture. The application of said dry mixture has the advantage of no special production or handling compared to fluid products (deodorants). The odor control material is incorporated in personal care products as a separate component from the superabsorbent material. Unfortunately, in such products, the absorption capacity of the superabsorbent polymer is reduced.

U.S. Pat. No. 4,795,482 and EP-B-0,297,543 disclose molecular sieves of the type SiO₂/Al₂O₃ having a molar ratio from 35 to infinity, preferably from 200 to 500, for use in odor control. At least 90% of the structure has to be built from the tetrahedral structure of silica. The average diameter of the pores should be at least 0.55 nm. Unfortunately, structures disclosed in these patents have only small absorption capacities of lower than 10% by weight (tested with water).

WO 98/28478 discloses layered structures of personal care products comprising hydrophilic fibers prepared and stabilized by addition of latices. Odor control material and superabsorbent material is added. Odor control compounds are, for example, disclosed in U.S. Pat. Nos. 3,093,546 (antimicrobial agents, e.g., halogenated phenylene), 4,385,632 (copper derivatives, preferably copper acetate), 4,525,410 (zeolite), 5,037,412 (acidic compounds such as ascorbic acid, stearic acid, citric acid, maleic acid, and polyacrylic acid). Mixtures of silica:zeolite:superabsorbent material are, for example, used in the ratio 1:5:1 to 1:1:5.

EP-A-0,894,502 relates to the use of α-cyclodextrin of particle size 12 to 800 μm as odor control material in an absorbent article containing a fluid-absorbing material, such as superabsorbent polymer particles or an absorbent foam. According to WO 00/66187, odor controlling superabsorbent polymer particles contain an odor controlling compound, such as cyclodextrin, triclosan, an amphoteric surfactant, a water-insoluble phosphate or mixtures thereof, homogeneously distributed throughout the particle.

As mentioned above, using odor control materials and superabsorbents separately leads to an absorption profile both for body fluids and odors which is substantially different from each compound used alone. Further, high loadings with an odor control material results in a reduced absorption profile of the superabsorbent polymers.

EP-A-0 341 951, U.S. Pat. Nos. 4,990,338, and 5,035,892 teach the addition of silica in preparation of antimicrobial polymers. Generally, the addition of silica to superabsorbent particles results in covering the surface area of the particles, and therefore influences only the properties of the surface of superabsorbent polymers, not the intrinsic absorption properties of the hydrogel particles. Thus, the addition of silica compounds leads to hydrophilation or hydrophobation of the superabsorbent particles and at first influences the absorption rate.

Despite optimizing the superabsorbent material, there still exists a phenomena called “gel blocking.” Gel blocking occurs when superabsorbent particles are wetted and the particles swell at the surface to inhibit fluid transmission to the inner part of the particle or to other regions of the absorbent structure. Wetting of other regions takes place by a very slow diffusion process. As a consequence, acquisition of aqueous body fluids by the absorbent structure is much slower than the rate at which the fluids are discharged, especially in gush situations. Leakage takes place before the superabsorbent particles are fully saturated, and before the fluids reach the unwetted regions.

Thus, the gel blocking phenomena necessitates the use of a matrix cellulosic fibers to serve as temporarily fluid storage reservoir. The aqueous body fluid is held in the pores of the fluff matrix. The superabsorbent particles are dispersed in the matrix, and thus separated from one another. The superabsorbent particles then absorb the fluid and dewater the fluff matrix. That is, the fluff serves to absorb and hold relatively large quantities of quickly applied body fluids until the superabsorbent material quantitatively absorbs the body fluid. From this point of view, it is impossible to create continuous layers of superabsorbent hydrogels.

Accordingly, a number of different structures have been proposed to overcome problems associated with incorporating superabsorbent materials in personal care products.

U.S. Pat. No. 4,699,619 describes a multilayer absorbent composite having a first relatively low density layer and a second relatively high density layer underlying at least a portion of the first layer. A high absorbency material (superabsorbent) is described as being located between said first and second layers.

U.S. Pat. No. 4,102,340 relates to a disposable article with a particulate hydrophilic polymer in an absorbent bed. Described therein is an absorbent pad comprising a fibrous structure having an intermediate densified layer and a layer of highly porous, loosely compacted bed on both sides of the densified layer. One of the bed layers is described as including a particulate, water-insoluble, but water-swellable polymeric absorbent.

U.S. Pat. No. 4,269,188 is directed to a disposable diaper. Disclosed therein is a disposable diaper including an absorbent material comprising a laminate wherein a water-absorbent polymer powder is fixed between two sheets of paper. A fluff pulp layer is located on both sides of the laminate containing a water-absorbing polymer.

While the structures described in the referenced patents often have proven beneficial, they have not completely solved problems associated with the use of superabsorbent materials. Thus, by using conventional methods, the wood pulp fluff serves to quickly absorb and hold fluid surges (relatively large quantities applied relatively quickly) of body fluid until the high-absorbency material can absorb the fluid. Then, the fluff matrix is dewatered by the high-absorbency material. Because of gel blocking, the ratio of superabsorbent to fiber remains too low for the intended use of absorbing large quantities of body fluids. Furthermore, the problem of separation of the incorporated, but not affixed, particulates by wearing the personal care product remains. Up to now, there is no proposed absorbent structure that overcomes the problem of incorporation higher amounts of superabsorbent materials without changing the absorption profile.

### SUMMARY OF THE INVENTION

It is desired to provide an absorbent structure which contains immobilized superabsorbent particles affixed to each other or onto a substrate. It is further desired to create an absorbent layer having a relatively high concentration of high absorbency material, but wherein an absorbent structure is capable of absorbing and storing body fluids applied thereto.

Substantial work was done to immobilize superabsorbent particles. One approach is the addition of large quantities of liquid polyhydroxy compounds that act as an adhesive to hold the particles together or attach the particles to a substrate. But during swelling, to some extent, the particles become detached from each other or from the substrate in the presence of excess liquid. Another immobilization technique is the formation of an interparticle crosslinked aggregate, wherein the aggregate is joined to a carrier, which may comprise cellulosic fibers or which may be formed by a web. Unfortunately interparticle crosslinking leads to loss of flexibility, and, therefore, an unpleasant feeling when wearing the absorbent member. Further, interparticle crosslinked aggregates tend to lose water when stored for a longer period of time, and thus the aggregates become stiff or brittle. More flexible absorbent structures are achieved by adhesive attachment of superabsorbent particles to fibers. Unfortunately, the adhesive attachment adversely influences the absorption profile of the particles and leads to nonuniform swelling.

EP-A-0 700 672, therefore, avoids adhesive attachment and proposes chemical crosslinking for joining superabsorbent particles to the web. The particles of mass median particle size of about 400 μm are applied to a substrate comprising crosslinking agent. Unfortunately, the basis weights of the superabsorbent particles are very low in order to prevent interparticle crosslinking and to obtain more flexibility of the composite. By attaching the superabsorbent particles to the substrate in an individual manner, there is no adverse influence on the absorption properties and no detachment of the particles by wetting. Moreover, the liquid transmission and flexibility of the substrate is increased. But the highly flexible composite structures comprising a relatively low absorbent capacity therefore are for use in products which are intended to absorb relatively small amounts of fluids, such as panty liners, sanitary napkins, or light incontinence pads.

EP-A-0 615 736 proposes containment means comprising two layers of material which are joined together to form a pocket or compartment. The pocket contains the superabsorbent material. Within the pockets, superabsorbent material is incorporated up to 100 wt % without any affixing onto the web. But, in order to prevent gel blocking, the hydrogel zone is interrupted by the formation of pockets. This proposal requires substantial effort in the manufacture of such absorbent members and therefore is very expensive.

WO 01/56625 relates to an acquisition layer in an absorbent article which is prepared by forming a sprayable blend comprising (a) one or more superabsorbent-forming monomers, (b) superabsorbent polymer particles, (c) water, and (d) one or more initiators, spraying said sprayable blend onto a fibrous web, and subjecting said fibrous web to conditions under which the superabsorbent forming monomer will polymerize. The absorbent articles are used in disposable hygiene products.

Generally, said web is loaded with superabsorbent material up to 60 or 70 wt % compared to conventional personal care products. It is desired to provide a matrix wherein superabsorbent polymer is incorporated in amounts convenient to have an optimum permeability and absorbency, and which exhibits wet strength integrity. The absorbent structure has to be of high flexibility and thinness, which therefore is very comfortable in use, even in wearing the personal care products over a long time period. Therefore, it is further desired to provide an absorbent structure having good odor control properties.

It is an object of the present invention to provide an absorbent structure which contains an absorbent layer having a relatively high concentration of high-absorbency material, but in which the absorbent structure is stable, and affixed to a matrix and cannot be detached by mechanical forces. It is desired to provide a matrix wherein the superabsorbent polymer is incorporated in amounts convenient to have an optimum absorbency and storage capacity, and which exhibits wet strength integrity. The new absorbent structure has to have a specific absorption capacity especially in regions where, in gush situations, the highest amounts of body fluids are applied.

It is further desired to provide an absorbent structure with an unchanged absorption profile after attachment of the superabsorbent material onto the substrate, which is capable of quickly absorbing body fluids applied thereto and afterwards storing them. Said superabsorbent polymer exhibits a highest absorption rate and absorption capacity without a tendency to gel block.

It is a further object of the invention to provide an absorbent structure of high flexibility and thinness, which therefore is very comfortable in use, even when wearing a personal care product over a long period of time.

It is further desired to provide an absorbent structure with an unchanged absorption profile after attachment of the superabsorbent material onto the substrate, which is capable of quickly absorbing body fluids applied thereto and afterwards storing them.

It is a further object of the invention to provide an absorbent structure of high flexibility and thinness, which is therefore very comfortable in use, even when wearing the personal care products over a long period of time and which exhibits odor control properties.

It is further object of the invention to provide an absorbent structure which can contain an absorbent layer having a relatively high concentration of high-absorbency material, but which absorbent structure is capable of quickly absorbing body fluids applied thereto, storing them, and which exhibits odor control properties. It is a further object of the invention to provide an absorbent structure which retains most of its absorption profile after the application of an odor control material.

The objects of the invention are achieved by the use of a layer obtainable by a process comprising

(a) forming a sprayable blend comprising one or more superabsorbent forming monomers, superabsorbent polymer particles, water, and one or more initiators;

(b) applying said sprayable blend on a fibrous web; and

(c) subjecting said fibrous web to conditions under which the superabsorbent forming monomer polymerizes as a storage layer, preferably by the use of such a layer in hygienic products.

Such a storage layer has a CRC equal to or greater than 9,000 g/m², preferably greater than 10,000 g/m², more preferably greater than 12,000 g/m².

The objects of the invention are achieved with an absorbent article comprising at least one double-sided coated fibrous web having a storage layer on one side and an acquisition layer on the other side and/or a combination of at least two adhering double-sided coated webs, with the proviso that one web has a storage layer on both sides and the other web has an acquisition layer on both sides wherein said layers are obtained by

(a1) forming a blend comprising one or more monomers capable of forming a superabsorbent polymer, superabsorbent polymer particles, water, and one or more initiators, said blend having a viscosity of at least 20 mPas (measured at 20° C. in a Brookfield viscometer, spindle 02, 20 rpm);

(b1) applying said blend onto a fibrous web; and

(c1) carrying out a polymerization of the monomers forming superabsorbent polymers.

The blend formed in step (a) further can contain a crosslinking agent and/or a softening agent and/or at least one odor control means and/or an agent with skin care effect, e.g., panthotenol, aloe vera, or a pH in the range of the skin. In the alternative, the superabsorbent polymer particles of the blend formed in step (a) can contain at least one odor control means and/or an agent with skin care effect, e.g., panthotenol, aloe vera, or a pH in the range of the skin.

The acquisition layer of the absorbent article has, for example, a pH of from 2.0 to 7.5, preferably from 4.0 to 6.5. In an preferred embodiment of the invention, the acquisition layer and the storage layer have different pH. For example, the pH of the acquisition layer may be from 4.0 to 6.5, preferably from 4.2 to 4.5, and the pH of the storage layer may be from 5.0 to 6.0.

In another preferred embodiment of the invention, the superabsorbent particles are mixed-bed ion exchange superabsorbent polymers or multidomain-composites of superabsorbent polymers, as described, for example, in WO 99/25393, preferably having an anionic to cationic superabsorbent ratio of from about 5:1 to about 1:5.

The objects of the invention also are achieved by a process for the production of an absorbent article which comprises

(a1) forming a blend comprising one or more monomers capable of forming superabsorbent polymers, superabsorbent polymer particles, water, and one or more initiators, said blend having a viscosity of at least 20 mPas (measured at 20° C. in a Brookfield viscometer, spindle 02, 20 rpm),

(b1) applying said blend onto both sides of at least one fibrous web,

(c1) subjecting said fibrous web to conditions under which the superabsorbent forming monomer polymerizes, and

(d1) combining by compression at least two coated webs which have different amounts of polymer coated thereon by carrying out steps (b1) and (c1).

The blend formed in step (a1) further may comprise a crosslinking agent and/or odor control means and/or a softening agent and/or skin care agent. If an odor controlling agent is added together with a constituent of the sprayable blend, it then is preferred to use odor control means containing superabsorbent polymer particles. The fibrous web of step (b1) also may contain one or more odor control means and/or skin care agents. It also is possible that the fibrous web, as well as the sprayable blend contain one or more odor controlling means.

The addition of one or more softening agents to the blend enables a production of soft and improved skin feel personal care products. Moreover, the whole absorbent web can be rendered to a soft absorbent structure of high flexibility with odor control properties. Nevertheless, said absorbent structure shows good absorbency and good strength. The improved webs retain most of the absorption profile of the absorbent polymers.

The absorbent article also may be prepared by

(a1) forming a blend comprising one or more monomers capable of forming superabsorbent polymers, superabsorbent polymer particles, water, and one or more initiators, said blend having a viscosity of at least 20 mPas (measured at 20° C. in a Brookfield viscometer, spindle 02, 20 rpm),

(b1) applying said blend onto one side of a fibrous web,

(c1) carrying out a polymerization of the monomers capable of forming superabsorbent polymers,

(d1) applying the blend formed in step (a1) to the other side of the web in a different amount, and

(e1) carrying out a polymerization of the monomers capable of forming superabsorbent polymer.

A higher polymer loading on the web, for example, more than 65% by weight, with reference to the weight of polymer and web, with up to about 100% by weight, results in storage layers, whereas a polymer loading produced by the process of the invention of less than 65% by weight, with reference to the polymer and web, results in an acquisition layer. Percentage by weight refers to the total weight of the dried layer with a residual water content of 0 to 5%, preferably 3%. In one embodiment of the above process, the blend formed in step (a) further may contain a crosslinking agent and/or a softening agent. In another embodiment of the process of the present invention, the superabsorbent polymer particles or the blend contain one or more odor control means.

The objects of the invention are achieved with an odor control agent containing absorbent article obtained by

(a2) forming a blend comprising one or more monomers capable of forming superabsorbent polymers, superabsorbent particles, water, one or more initiators, said blend having a viscosity of at least 20 mPas (measured at 20° C. in a Brookfield viscometer, spindle 02, 20 rpm),

(b2) applying said blend onto a fibrous web, and

(c2) carrying out a polymerization of the monomers capable of forming superabsorbent polymers in the presence of at least one odor control means, which may be present in either of the blend formed in step (a2) and/or the fibrous web, by subjecting said fibrous web to conditions under which the monomers capable of forming superabsorbent polymers polymerize or polymerizing the blend on the fibrous web and adding thereafter an odor control agent to the absorbent article.

The objects also are achieved with an absorbent article which is obtained by carrying out the above steps (a2) to (c2), when the blend formed in step (a2) further comprises a crosslinking agent.

The odor-control means can be present in the blend formed in step (a2), i.e., one or more odor control agents are added to the sprayable blend and/or may be contained in the superabsorbent polymer particles or in the softening agent. If an odor controlling agent is added together with a constituent of the sprayable blend, it then is preferred to use odor control means containing superabsorbent polymer particles. The fibrous web of step (b2) also may contain one or more odor control means. It also is possible that the fibrous web as well as the sprayable blend contain one or more odor controlling means.

The obtained absorbent structure has a capability of effectively controlling odors related to absorbed fluids. The addition of one or more softening agents enables production of soft and improved skin feel personal care products. Moreover, the whole absorbent web can be rendered to provide a soft absorbent structure of high flexibility with odor control properties. Nevertheless, said absorbent structure shows good absorbency and good strength. The improved webs retain most of the absorption profile of the absorbent polymers.

The odor control agent-containing absorbent article may be prepared by

(a2) forming a blend comprising one or more monomers capable of forming superabsorbent polymers, superabsorbent polymer particles, water, and one or more initiators, said blend having a viscosity of at least 20 mPas (measured at 20° C. in a Brookfield viscometer, spindle 02, 20 rpm),

(b2) spraying said sprayable blend onto a fibrous web, and

(c2) carrying out a polymerization of the monomers capable of forming superabsorbent polymers in the presence of at least one odor control means, which may be present in either of the blend formed in step (a2) and/or the fibrous web, by subjecting said fibrous web to conditions under which the monomers capable of forming superabsorbent polymers polymerize or polymerizing the blend on the fibrous web and adding thereafter an odor control agent to the absorbent article.

In one embodiment of the above process, the sprayable blend formed in step (a2) further comprises a crosslinking agent. In another embodiment of the process of the present invention, the superabsorbent polymer particles contain one or more odor control means.

### DETAILED DESCRIPTION OF THE INVENTION

Monomers Forming Superabsorbent Polymers

Superabsorbent polymer-forming monomers, as used herein, are polymerizable compounds which contribute to the absorbency of the polymers formed therefrom. Suitable monomers forming superabsorbent polymers useful in the present invention include monoethylenically unsaturated compounds (or compounds having a polymerizable double bond), having at least one hydrophilic radical, such as carboxyl, carboxylic acid anhydride, carboxylic acid salt, sulfonic acid, sulfonic acid salt, hydroxyl, ether, amide, amino or quaternary ammonium salt groups. Examples of suitable monomers forming superabsorbent polymers are as follows:

1. Carboxyl group-containing monomers: monoethylenically unsaturated mono- or polycarboxylic acids, such as (meth)acrylic acid (meaning acrylic acid or methacrylic acid, similar notations are used hereinafter), maleic acid, fumaric acid, crotonic acid, sorbic acid, itaconic acid, and cinnamic acid;

2. Carboxylic acid anhydride group-containing monomers: monoethylenically unsaturated polycarboxylic acid anhydrides (such as maleic anhydride);

3. Carboxylic acid salt-containing monomers: water-soluble salts (alkali metal salts, ammonium salts, amine salts, etc.) of monoethylenically unsaturated mono- or polycarboxylic acids (such as sodium (meth)acrylate, trimethylamine (meth)acrylate, triethanolamine (meth)acrylate, sodium maleate, and methylamine maleate);

4. Sulfonic acid group-containing monomers: aliphatic or aromatic vinyl sulfonic acids (such as vinylsulfonic acid, allylsulfonic acid, vinyltoluenesulfonic acid, styrenesulfonic acid), (meth)acrylic sulfonic acids (such as sulfopropyl (meth)acrylate, 2-hydroxy-3-(meth)acryloxy propyl sulfonic acid);

5. Sulfonic acid salt group-containing monomers: alkali metal salts, ammonium salts, amine salts of sulfonic acid group-containing monomers as mentioned above;

6. Hydroxyl group-containing monomers: monoethylenically unsaturated alcohols (such as (meth)allyl alcohol), monoethylenically unsaturated ethers or esters of polyols (alkylene glycols, glycerol, and polyoxyalkylene polyols), such as hydroxyethyl (meth)acrylate, hydroxypropyl (meth)acrylate, triethylene glycol (meth)acrylate, and poly(oxyethylene oxypropylene) glycol mono (meth)allyl ether (in which hydroxyl groups may be etherified or esterified);

7. Amide group-containing monomers: vinylformamide, (meth)acrylamide, N-alkyl (meth)acrylamides (such as N-methylacrylamide and N-hexylacrylamide), N,N-dialkyl (meth)acrylamides (such as N,N-dimethylacrylamide and N,N-di-n-propylacrylamide), N-hydroxyalkyl (meth)acrylamides (such as N-methylol(meth)acrylamide and N-hydroxyethyl(meth)acrylamide), N,N-dihydroxyalkyl (meth)acrylamides (such as N,N-dihydroxyethyl(meth)acrylamide), and vinyl lactams (such as N-vinylpyrrolidone);

8. Amino group-containing monomers: amino group-containing esters (e.g., dialkylaminoalkyl esters, dihydroxyalkylaminoalkyl esters, and morpholinoalkyl esters) of monoethylenically unsaturated mono- or dicarboxylic acid (such as dimethylaminoethyl (meth)acrylate, diethylaminoethyl (meth)acrylate, morpholinoethyl (meth)acrylate, and dimethyl aminoethyl fumarate), heterocyclic vinyl compounds, such as vinyl pyridines (e.g., 2-vinyl pyridine, 4-vinyl pyridine, and N-vinyl pyridine) and N-vinyl imidazole; and

9. Quaternary ammonium salt group-containing monomers: N,N,N-trialkyl-N-(meth)acryloyloxyalkylammonium salts (such as N,N,N-trimethyl-N-(meth)acryloyloxyethylammonium chloride, N,N,N-triethyl-N-(meth)acryloyloxyethylammonium chloride, 2-hydroxy-3-(meth)acryloyloxypropyl trimethyl ammonium chloride), and monomers as mentioned in British patent specification No. 1,034,296.

Suitable monomers which become water-soluble by hydrolysis for use in this invention, instead of or in conjunction with the water-soluble monomers, include monoethylenically unsaturated compounds having at least one hydrolyzable group, such as esters, amide, and nitrile groups. Monomers having an ester group include for example, lower alkyl (C₁-C₄) esters of monoethylenically unsaturated carboxylic acids, such as methyl (meth)acrylate, ethyl (meth)acrylate and 2-ethylhexyl (meth)acrylate; and esters of monoethylenically unsaturated alcohols (vinyl esters, (meth)allyl ester, etc.), such as vinyl acetate and (meth)allyl acetate. Suitable nitrile group-containing monomers include (meth)acrylonitrile.

Among these monomers having a polymerizable double bond which are water-soluble or become water-soluble by hydrolysis, water-soluble monomers that do not need hydrolysis after polymerization are preferred from the viewpoint of providing an easy process for producing water-absorbing resins. Further, from the viewpoint of providing water-absorbing resins having higher water-absorbance, the preferred water-soluble monomers are carboxyl group-containing monomers such as (meth)acrylic acid and maleic acid anhydride; carboxyl acid salt group-containing monomers such as sodium (meth)acrylate, trimethylamine (meth)acrylate, and triethanolamine (meth)acrylate; and quaternary ammonium salt group-containing monomers, such as N,N,N-trimethyl-N-(meth)acryloyloxyethylammonium chloride. Most preferred superabsorbent forming monomers in the present invention include, for example, acrylic acid, methacrylic acid, maleic acid, fumaric acid, crotonic acid, sorbic acid, itaconic acid, cinnamic acid, vinylsulfonic acid, allylsulfonic acid, vinyltoluene sulfonic acid, styrenesulfonic acid, sulfo(meth)acrylate, sulfopropyl(meth)acrylate, 2-acrylamido-2-methylpropane sulfonic acid, 2-hydroxyethyl (meth)acryloylphosphate, phenyl-2-acryloyloxyethylphosphate, the sodium, potassium and ammonium salts thereof, maleic anhydride, and combinations thereof. It also is preferred that the superabsorbent forming monomer in the sprayable blend is at least partially neutralized, preferably neutralized to a level of from 1 to 100 mole percent, more preferably from 10 to 80 mole percent, and most preferably from 15 to 75 mole percent. Most preferably, the superabsorbent forming monomer is neutralized acrylic acid.

The monomers forming superabsorbent polymers are present in the blends formed in step (a) at a level of from about 15 to 50 percent by weight, preferably from 17 to 40 percent by weight, most preferably from 20 to 35 percent by weight. If the level of the monomers forming superabsorbent polymers in said blend is too low, the resulting web may have poor performance characteristics. Within the preferred ranges, the conversion of superabsorbent polymer-forming monomers into polymer, when subjected to polymerization conditions, is generally much better. Also, increasing the relative amount of monomers forming superabsorbent polymers in the blend formed in step (a) generally reduces the amount of water in the blend. This is beneficial because it requires time, energy, and expense to remove additional water from the final web. When partially neutralized acrylic acid is used as monomer forming superabsorbent polymers, e.g., 75% neutralized, it is preferred to use the partially neutralized acrylic acid in the blend in a concentration of no more than 38% by weight.

Superabsorbent Polymer Particles

Superabsorbent polymer particles are lightly crosslinked polymers capable of absorbing several times their own weight in water and/or saline. Superabsorbent polymer particles can be made by any conventional process for preparing superabsorbent polymers and are well known to those skilled in the art. Suitable process for preparing superabsorbent polymer particles include the processes described in U.S. Pat. Nos. 4,076,663; 4,286,082; 4,654,039; and 5,145,906, which describe the solution polymerization method, and U.S. Pat. Nos. 4,340,706; 4,497,930; 4,666,975; 4,507,438; and 4,683,274, which describe the inverse suspension polymerization method, the disclosures of which are hereby incorporated by reference. Preferred superabsorbent polymer particles have an average particle size small enough such that the particles do not clog spray equipment, preferably below about 150 μm, more preferably below about 100 μm. Such particle size can be obtained directly as a result of the polymerization process, or superabsorbent polymers can be sieved, ground, pulverized, attritted, or a combination thereof to achieve superabsorbent polymer particles having the desired average particle size. The mean particle size diameter of the superabsorber polymer particles is, for example, in the range from 10 to 130 μm, preferably 15 to 100 μm, and most preferably 40 to 90 μm.

The superabsorbent polymer particles are present in the blends formed in step (a1) or (a2) at a level of from about 1 to 20 percent by weight, preferably from 2 to 15 percent by weight, and most preferably from 5 to 10 percent by weight. It has been observed that if the level of superabsorbent polymer particles is too high, premature polymerization can occur in the blend even in the absence of an initiator.

Superabsorbent polymer particles useful in the present invention are prepared from one or more monoethylenically unsaturated, water-soluble carboxyl or carboxylic acid anhydride containing monomers, and the alkali metal and ammonium salts thereof, wherein said monomers comprise 50 to 99.9 mole percent of said polymer. Exemplary monomers include acrylic acid, methacrylic acid, maleic acid, fumaric acid, maleic anhydride, and the sodium, potassium, and ammonium salts thereof. The preferred monomer is acrylic acid.

Monoethylenically unsaturated monomers are polymerized in the presence of an internal crosslinking compound to provide a lightly crosslinked base polymer wherein crosslinking is substantially uniform throughout the polymer particles. These internal crosslinkers are well known in the art. Suitable crosslinkers are those compounds having two or more groups capable of reacting with the monoethylenically unsaturated monomers and which are at least partially water soluble or water dispersible, or at least partially soluble or dispersible in an aqueous monomer mixture. The internal crosslinking compound may be selected from a polyunsaturated monomer such as divinylbenzene, a compound having at least two functional groups which are reactive with the monoethylenically unsaturated monomer, such as ethylendiamine, and a compound having at least one unsaturated bond and at least one reactive functional group, such as glycidyl acrylate.

Exemplary internal crosslinkers are: tetraallyloxyethane, N,N′-methylene bisacrylamide, N,N′-methylene bismethacrylamide, triallylamine, trimethylol propane triacrylate, glycerol propoxy triacrylate, divinylbenzene, N-methylol acrylamide, N-methylol methacrylamide, glycidyl methacrylate, polyethylene polyamines, ethyl diamine, ethyl glycol, glycerine, and the like. Preferred internal crosslinking monomers are those containing at least two allyl groups, most preferably three or four allyl groups. Preferred internal crosslinkers are tetraallyloxyethane and the triallyl ether of penta-erythritol. The amount of internal crosslinker employed in the invention will depend on the internal crosslinker and the polymerization method. Generally the amount of internal crosslinker will vary from about 0.005 to about 1.0 mole percent based on moles of ethylenically unsaturated monomer.

Optional components used in the preparation of the superabsorbent polymer particles are water soluble hydroxy group containing polymers, such as polysaccharides and vinyl or acrylic polymers. Examples of water soluble polysaccharides are starches, water soluble celluloses, and polygalaactomannans. Suitable starches include the natural starches, such as sweet potato starch, potato starch, wheat starch, corn starch, rice starch, tapioca starch, and the like. Processed or modified starches, such as dialdehyde starch, alkyl-etherified starch, allyl-etherified starch, oxyalkylated starch, aminoethyl-etherified starch, and cyanomethyl-etherified starch also are suitable. Polyvinyl alcohol and polyvinyl alcohol copolymers also are suitable.

The water-soluble celluloses useful in this invention are those obtained from such sources as wood, stems, bast, seed fluffs, and the like, which then are deriviatized to form hydroxyalkylcellulose, carboxymethylcellulose, methylcellulose, and the like.

Suitable polygalactomannans are guar gum and locust bean gums, as well as their hydroxyalkyl, carboxyalkyl, and aminoalkyl derivatives. Water soluble vinyl and acrylic polymers include polyvinyl alcohol and poly(hydroxyethyl acrylate). The preferred polysaccharide for use in this invention is natural starch, such as wheat starch, corn starch, and alpha starches. These optional preformed hydroxy containing polymers may be used in an amount from about 1 to 15 percent, preferably about 1 to 10 percent, and most preferably about 1 to 5 percent.

The superabsorbent polymer particles useful in the present invention may be prepared by well known polymerization methods. The polymerization reaction is conducted in the presence of, for example, redox initiators and thermal initiators. The redox initiators can be used as the primary initiator with the thermal polymerization initiators being used if desired to reduce the free monomer content of the final polymerization product below 0.1 percent by weight. Optionally, thermal initiators or redox initiators may be used as the sole initiator system. Examples of different initiator systems are found in U.S. Pat. No. 4,497,930, which discloses a two component initiator system comprising a persulfate and a hydroperoxide, and U.S. Pat. No. 5,145,906, which discloses a three component initiator system, i.e., redox system plus thermal initiator.

Any of the well known water soluble reducing agents and oxidizing agents can be used in this invention as the redox initiator. Examples of reducing agents include such compounds as ascorbic acid, alkali metal sulfites, alkali metal bisulfites, ammonium sulfite, ammonium bisulfite, alkali metal hydrogen sulfite, ammonium hydrogen sulfite, ferrous metal salts, e.g., ferrous sulfates, sugars, aldehydes, primary and secondary alcohols, and the like.

Oxidizing agents include such compounds as hydrogen peroxide, alkali metal persulfates, ammonium persulfates, alkylhydroperoxides, peresters, diacryl peroxides, silver salts, and the like. A particularly preferred redox initiator pair is ascorbic acid and hydrogen peroxide. The reducing agent is used, for example, in an amount of about 2×10⁻⁵ to about 2×10⁻² mole percent based on moles of acrylic acid.

In order to ensure complete polymerization of the unsaturated monomer and the crosslinking monomer, a thermal initiator may be included in the polymerization process. Useful thermal initiators are the “azo” initiators, i.e., compounds which contain the —N═N— structure. Any of the azo compounds which have solubility in water or in a monomer-water mixture and which have a ten hour half-life at 30° C. or above can be used. Examples of useful azo initiators are 2,2′-azobis(amidino)propane dihydrochloride, 4,4′-azobis(cyanovaleric acid), 4,4′-butylazocyanovaleric acid, 2,2′-azobis(isobutyronitrile), 2,2′-azobis(2-(2-imidazole-2-yl))propane dihydrochloride, and the like. Other thermal initiators include the persulfates and hydroperoxides when used in the absence of a reducing agent, e.g., sodium, potassium and ammonium persulfates, t-butylhydroperoxide, and the like. A preferred azo initiator for use in this invention is 2,2′-azobis(amidino)propane dihydrochloride. The thermal initiators are used in the amount of 0 to about 1 mole percent based on the weight of unsaturated monomer.

The superabsorbent polymer may be prepared by the solution or the inverse suspension polymerization method or any suitable bulk polymerization method. The solution polymerization and inverse polymerization methods are well known in the art; see for example U.S. Pat. Nos. 4,076,663; 4,286,082; 4,654,039; and 5,145,906, which describe the solution polymerization method, and U.S. Pat. Nos. 4,340,706; 4,497,930; 4,666,975; 4,507,438; and 4,683,274, which describe the inverse suspension polymerization method. The teachings of these patents are hereby incorporated by reference.

In the solution polymerization method, the water soluble monomer is polymerized at a monomer concentration from about 5 to about 30 percent in aqueous solution at a temperature from about 5° C. to about 150° C. depending upon the polymerization initiator system. A detailed description of the solution polymerization method is given in U.S. Pat. No. 5,145,906, the teachings of which are hereby incorporated by reference.

In the inverse suspension polymerization process, the unsaturated monomer in an aqueous solution (about 35 to 60 percent monomer and 65 to 40 percent water) is dispersed in an alicyclic or aliphatic hydrocarbon suspension medium in the presence of a dispersing agent, such as a surfactant or protective colloid, such as polyvinyl alcohol. A surfactant having a HLB value of 8 to 12, such as a sorbitan fatty acid ester, may be employed as the dispersing agent. The inverse suspension polymerization method is described in detail in U.S. Pat. No. 4,340,706, the teachings of which are hereby incorporated by reference.

The carboxylic acid groups of the unsaturated monomer used in the polymerization may be neutralized prior to or subsequent to the polymerization. Suitable neutralizing agents include an alkali, such as sodium hydroxide, ammonium hydroxide, potassium hydroxide, or the like, and the appropriate degree of neutralization is 50-98 mole percent, preferably 60-75 mole percent. The degree of neutralization preferably is at least 50 mole percent. Low neutralization levels (less than 50 mole percent) tend to result in superabsorbent polymers having lower absorbency properties.

The polymer is prepared by either the solution or inverse suspension polymerization method, dried, and screened to provide a superabsorbent particle with an appropriate particle size distribution and particle shape. Generally, the superabsorbent particle size distribution (mean particle diameter) should be between 10 and 300 μm, preferably between 45 and 150 μm and most preferably 50 to 90 μm. Large particles over 300 μm are undesired because they tend to clog the nozzle of spray equipment used to spray the sprayable blend. Also, large particles may cause the absorbent article to have an abrasive feel.

Although many of the conventional teachings in this area prefer to avoid the use of small particle size superabsorbent polymer particles, it has been found that these particles work quite well in the present invention. Superabsorbent polymer particles having particle sizes in the range of from 10 to 300 μm tend to work well with the spray equipment, produce sprayable blends having manageable viscosities, and result in absorbent articles having discrete superabsorbent particles attached to the fibrous web.

The superabsorbent polymer particles described above optionally may be further treated with a crosslinker solution containing from about 0.5 to about 3.5 weight percent water, from about 1.0 to 2.5 weight percent of a water miscible solvent selected from a C₃-C₆ diol, and a crosslinker having at least two functional groups that can react with the carboxyl, carboxylate, or other reactive groups in the superabsorbent polymer chain to crosslink the polymer chains on or in the vicinity of the surface of superabsorbent polymer particles.

The term “diol” is intended to mean a dihydroxy aliphatic compound which may be a linear or branched compound, i.e., a glycol. The term “surface crosslinking” is used to describe this process of crosslinking the polymer chains on or in the vicinity of the particle's surface. The terms, “surface crosslinker” and “surface crosslinker solution” are likewise used to describe the crosslinking compound and the solution used to effect this surface crosslinking process.

The crosslinking compound is used, for example, in an amount of from about 0.01 to about 3 weight percent, preferably 0.1 to 1.5 weight percent, and most preferably 0.25 to 1 weight percent, based upon the weight of the superabsorbent polymer. The surface crosslinker may be selected from diglycidyl ethers, haloepoxy, isocyanate, aziridinyl, azetidinium group containing compounds, oxazolidone, polyamine, polyamine-polyamide, polyamine-epichlorohydrin adducts, amine-polymer-epichlorohydrin adducts, and the like. Preferred crosslinkers are diglycidyl ether compounds having a molecular weight of at least 200 and the polymeric epichlorohydrin adducts having a molecular weight average in excess of 2000.

Exemplary surface crosslinkers are poly(ethylene glycol) diglycidyl ethers, poly (propylene glycol) diglycidyl ethers, epichlorohydrin substituted compounds, methyl-epichlorohydrin substituted compounds, hexamethylene diisocyanate, triethylene triamine, polyethylene amine, 2-oxazolidone, 2,2-bishydroxymethylbutanol-tris(3-(1-azindinyl)propionate), polyamine epichlorohydrin adducts, polyethylene-polyamine-epichlorohydrin adducts, and the like.

When used, the preferred surface crosslinkers are the higher molecular weight diglycidyl ether compounds, polyamide (polyamide-polyamine) epichlorohydrin adducts, polyamine-epichlorohydrin adducts, and amine polymer epichlorohydrin adducts. Polyamide-epichlorohydrin adducts are prepared by reacting epichlorohydrin with the polycondensation product of a polyalkylene polyamine with a polycarboxylic acid, such as diethylene triamine with a dibasic acid, like adipic acid. Polyamine-epichlorohydrin adducts are made by condensing a polyalkylene polyamine directly with epichlorohydrin. These adducts include polyalkylene polyamines which are linked together with dihalides to form higher polyamines before reacting them with epichlorohydrin. Amine polymer epichlorohydrin adducts include resins in which the monomeric amine is polymerized to a polyamine precursor which is then alkylated and reacted with epichlorohydrin. They include amines substituted polymers of vinyl, allyl, acrylate, or epoxy monomers. The epichlorohydrin adducts, whether the polymer is polyamide, a polyamine, or an amine polymer, react with the epichlorohydrin by different routes. If the amino group in the polymer chain is a primary amine, two epichlorohydrin molecules react with the nitrogen and form a disubstituted chlorohydroxypropyl substituted amine group. Secondary amine groups react with epichlorohydrin to form a tertiary aminochlorohydrin group which gives a reactive 3-hydroxyazetidinium salt moiety. This is a preferred reactive group. Tertiary amine groups react with epichlorohydrin to form a glycidyl (2,3-epoxypropyl) ammonium salt. Preferably, the reactive group is an azetidinium moiety. However, these adducts may contain a mixture of chlorohydroxypropyl, epoxy, and azetidinium groups. Preferably, the epichlorohydrin adducts have a molecular weight of at least 2,000, more preferably 300,000 to 500,000, and wherein at least 50 mole percent of the reactive groups in the adduct are the azetidinium groups. A preferred polymer is one in which about 90% of the substitution is in the form of an azetidinium group and about 10% as an epoxide group. Exemplary products are RETEN RTM.204LS and KYMENE RTM.736 epichlorohydrin adducts, available from Hercules Inc., Wilmington, Del. These products are sold as an aqueous solution of the reactive epichlorohydrin adduct. The RETEN RTM.204LS product is a 38% aqueous solution.

The surface crosslinker solution should have a surface tension not greater than about 55 dynes per cm, preferably not greater than about 50 dynes per cm, e.g., about 40 to 50 dynes per cm. In the event the surface tension of the crosslinker solution is higher than about 55 dynes per cm, the surface crosslinked polymer has inferior absorbency as evidenced by a low 0.6 psi AUL value. While not being bound to any theory, it is believed that when the surface tension of the crosslinker solution is higher than about 55 dynes per cm, the solution is not uniformly distributed on the surface of the polymer particles and a lower absorbency value results. Optionally, a surfactant may be used to reduce the surface tension of the crosslinker solution.

The desired surface tension is achieved by adding the C₃ to C₆ dihydroxy compound to the water component of the crosslinker solution to achieve a surface tension below about 55 dynes per cm range. The amount of each solvent is determined by simple experimentation. Generally the crosslinker has a negligible effect on the surface tension of the crosslinker solution. The diols useful in the invention are propylene glycol, butylene glycol, pentanediol, and hexanediol. Ethylene glycol was found to be an undesired solvent because it tends to swell the superabsorbent polymer particles, and their surfaces becomes tacky which results in undesired particle agglomeration. In addition, ethylene glycol is undesirable because of its toxicity and biodegradability properties. The C₃ to C₆ diol is used in an amount of from about 1 percent by weight to about 2.5 percent by weight based upon the weight of superabsorbent polymer, preferably about 1 to about 2 percent by weight. The water component of the crosslinker solution comprises about 0.5 to 3.5 percent by water based upon the weight of the polymer, preferably about 1.5 to 2.0 percent.

The total amount of crosslinker solution used depends upon the type of equipment and the method used to coat the base polymer with the surface crosslinking solution. Generally the amount of crosslinker solution should be about 1.5% minimum based of the weight of the polymer. The crosslinker solution is applied to the base polymer particles in a manner such that the solution is uniformly distributed on the surface of the base polymer particle. Any of the known methods for dispersing a liquid can be used, preferably by dispersing the crosslinker solution into fine droplets, e.g., by use of a pressurized nozzle or a rotating disc. Uniform crosslinker dispersion on the base polymer can be achieved in a high intensity mechanical mixer or a fluidized mixture which suspends the base polymer in a turbulent gas stream. Methods for the dispersion of a liquid onto the superabsorbent base polymer surface are known in the art, see, for example, U.S. Pat. No. 4,734,478, the teachings of which are hereby incorporated by reference, in particular column 6, line 45 to column 7, line 35.

Exemplary commercially available equipment for conducting the crosslinker solution dispersion step of the invention are high speed variable intensity paddle mixers such as the “Turbulizer” mixer of the Bepex Corporation, Rolling Meadows, Ill., or the high speed variable intensity vertical mixer sold by Bepex under the tradename, “Turboflex.” These machines generally are operated in a continuous manner using a short residence time in the order of 2 seconds to 2 minutes, typically 2-30 seconds. Dispersion may be effected batchwise in a high intensity mixer, such as a Henschel mixer, or in liquid-solid V-blender equipped with a liquid dispersion device. In any event, whether a batchwise or continuous dispersion method is used, simple experimentation can be conducted to determine the best process conditions for the particular machine employed in the process. Preferably, the surface crosslinker is coated onto the polymer particles under high intensity mixing conditions.

After effecting dispersion of the surface crosslinker on the base polymer particle, the crosslinking reaction is effected and the polymer partide dried. The crosslinking reaction may be effected at a temperature from about 70° C. to about 220° C., preferably to about 180° C.

In a preferred embodiment of the invention, a mixture of cationic and anionic hydrophilic absorbent polymers (“bipolar absorbent polymers”) are used. Such polymers generally are obtained by mixing cation-exchange absorbent polymers with anion-exchange absorbent polymers. Cation-exchange polymers are, for example, ethylene maleic anhydride copolymer, crosslinked polyvinyl sulphonic acid, crosslinked polyacrylic acid, or the graft copolymers of said monomers with starch or cellulose based polymer. The functional groups are selected from sulphonic, sulfate, phosphonates, or carboxyl. Preferred cation-exchange based polymers are crosslinked polyacrylates or crosslinked isobutylene maleic anhydride copolymer. Anion-exchange polymers contain primary, secondary, or tertiary amine or quaternary ammonium groups, for example, polyvinylamine, polyallylamine, polyethyleneimine, and polydiallyldimethyl ammonium hydroxide. The superabsorbent particles have a anionic to cationic superabsorbent ratio from about 5:1 to about 1:5. Such products are, for example, disclosed in U.S. Pat. No. 6,222,091 and WO 98/37149, both incorporated herein by reference.

Odor Control Means

In one embodiment of the invention, superabsorbent polymer particles contain odor control means. Any of the odor control materials known in the art can be used. Further, it is possible to use mixtures thereof. Odor control materials known in the art include zeolites, silica, carbon, chelants, antimicrobial agents, perfuming ingredients, masking agents, and mixtures thereof, for example:

Inorganic materials for odor control and absorption at the same time (e.g., zeolites, activated carbon, bentonite, silica (AEROSIL® or CAB-O-SIL®), aerosil, and clays) have relatively high surface areas. They are added in form of powder or granules to the surface of superabsorbent polymer particles.

Chelants prevent malodors by complexing organic molecules or metal ions present in body fluids. Preferably ethylenediaminetetraacetic acid or cyclodextrin (alpha-cyclodextrin, beta-cyclodextrin, gamma-cyclodextrin and/or their derivatives and/or mixtures thereof) are used. Most preferably mixtures of cyclodextrins are used. Generally, cyclodextrins may have particle sizes between 12 to 150 μm, or are added in particle sizes of lower than 12 μm to have highest surface areas for odor control. Further chelants are aminopolycarbonic acids and their salts, ethylenediaminepentamethylene phosphonic acid, ethylenediaminetetramethylene phosphonic acid, aminophosphate, polyfunctional aromates, and N,N-disuccinic acid.

Nonlimiting examples of perfuming ingredients, including preferred volatile perfume ingredients, are allo-ocimene, allyl caproate, allyl cyclohexaneacetate, allyl cyclohexanepropionate, allyl heptanoate, amyl acetate, amyl propionate, anethol, anixic aldehyde, anisole, benzaldehyde, benzyl acetate, benzyl acetone, benzyl alcohol, benzyl butyrate, benzyl formate, benzyl isovalerate, benzyl propionate, α-γ-hexenol, butyl benzoate, butyl caproate, 4-tert-butylcyclohexyl formate, camphene, camphor gum. carvacrol, levo-carveol, d-carvone, levo-carvone, cinnamyl formate, cis-jasmone, cis-3-hexenyl acetate, cis-3-hexenyl butyrate, cis-3-hexenyl caproate, cis-3-hexenyl tiglate, cis-3-hexenyl valerate, citral, citronellol, citronellyl acetate, citronellyl formate, citronellyl isobutyrate, citronellyl nitrile, citronellyl oxyacetaldehyde, citronellyl propionate, cuminic alcohol, cuminic aldehyde, Cyclal C, cyclohexyl ethylacetate, α-damascone, 2-decenal, decyl aldehyde, dihydro myrcenol, dihydro myrcenyl acetate, dimethyl benzyl carbinol, dimethyl benzyl carbinyl acetate, dimethyl benzyl carbinyl propionate, dimethyl phenylethyl carbinyl acetate, 3,7-dimethyloctanal, dimethyloctanol, diphenyl oxide, ethyl acetate, ethyl acetoacetate, ethyl amyl ketone, ethyl benzoate, ethyl butyrate, ethyl hexyl ketone, ethyl phenyl acetate, eucalyptol, fenchyl acetate, fenchyl alcohol, α-methyl-ionone, α-nonalactone, geraniol, geranyl acetate, geranyl acetoacetate, geranyl butyrate, geranyl formate, geranyl isobutyrate, geranyl nitrile, geranyl propionate, heptyl acetate, heptyl isobutyrate, heptyl propionate, hexenol, hexenyl acetate, hexenyl isobutyrate, hexyl acetate, hexyl formate, hexyl isobutyrate, hexyl isovalerate, hexyl neopentanoate, hexyl tiglate, hydratropic alcohol, hydroxy citronellal, α-ionone, β-ionone, γ-ionone, α-irone, isoamyl alcohol, isobornyl acetate, isobornyl propionate, isobutyl benzoate, isobutyl caproate, isononyl acetate, isononyl alcohol, isomenthol, isomenthone, isononyl acetate, isopulegol, isopulegyl acetate, isoquinoline, lauric aldehyde, lavandulyl acetate, ligustral, d-limonene, linalool, linalool oxide, linalyl acetate, linalyl butyrate, linalyl isobutyrate, linalyl formate, linalyl propionate, menthone, menthyl acetate, methyl acetophenone, methyl amyl ketone, methyl anthranilate, methyl benzoate, methyl benzyl acetate, methyl chavicol, methyl eugenol, methyl heptenone, methyl heptine carbonate, methyl heptyl ketone, methyl hexyl ketone, methyl nonyl acetaldehyde, α-iso“γ”-methyl ionone, methyl octyl acetaldehyde, methyl octylketone, methyl phenyl carbinyl acetate, methyl salicylate, myrcene, myrcenyl acetate, neral, nerol, neryl acetate, nonalactone, nonyl butyrate, nonyl alcohol, nonyl acetate, nonyl aldehyde, octalactone, octyl acetate, octyl alcohol, octyl aldehyde, orange terpenes, p-cresol, p-cresyl methyl ether, p-cymene, p-isopropyl-p-methyl acetophenone, phenethyl anthranilate, phenoxy ethanol, phenyl acetaldehyde, phenyl ethyl acetate, phenyl ethyl alcohol, phenyl ethyl dimethyl carbinol, α-pinene, β-pinene, prenyl acetate, propyl butyrate, pulegone, rose oxide, safrole, α-terpinene, γ-terpinene, 4-terpineol, terpineol, terpinolene, terpinyl acetate, terpinyl propionate, tetrahydro linalool, tetrahydro myrcenol, thymol, tricyclo decenyl acetate, tricyclo decenyl propionate, δ-undecalactone, γ-undecalactone, undecanal, undecenal, undecyl alcohol, Veratrol, Verdox, Vertenex, viridine.

The antimicrobial agent may be any chemical capable of preventing the growth of or killing microorganisms, and is capable of preferably binding to the surface superabsorbent material. Preferred antimicrobials are those that can prevent the growth of or kill microorganisms typically found in body fluids, more preferably those body fluids typically collected by a disposable absorbent article. Preferred antimicrobials include, but are not limited to, quaternary ammonium, phenolic, amide, acid, and nitro compounds, and mixtures thereof; more preferably quaternary ammonium, acid, and phenolic compounds; more preferably still quaternary ammonium compounds. Preferred quaternary ammonium compounds include, but are not limited to, 2-(3-anilinovinyl-ul)-3,4-dimethyloxazolinium iodide, alkylisoquin-olium bromide, benzalkonium chloride, benzethonium chloride, cetylpyridinium chloride, chlorhexidine gluconate, chlorhexidine hydrochloride, lauryl trimethyl ammonium compounds, methylbenzethonium chloride, stearyltrimethylammonium chloride, and mixtures thereof; more preferably benzalkonium chloride, chlorhexidine gluconate, and 3-(trimethoxysilyl)propyldimethyloctadecyl ammonium chloride.

Preferred phenolic compounds include, but are not limited to, benzyl alcohol, p-chlorophenol, chlorocresol, chloroxylenol, cresol, o-cymene-5-ol (BIOSOL), hexachlorophene, hinokitiol, isopropyl-methylphenol, parabens (having methyl, ethyl, propyl, butyl, isobutyl, isopropyl, and/or sodium methyl substituents), phenethyl alcohol, phenol, phenoxyethanol, o-phenylphenol, resorcin, resorcin monoacetate, sodium parabens, sodium phenolsulfonate, thioxolone, 2,4,4′-trichloro-2′-hydroxidiphenyl ether, zinc phenolsuflonate, di-tert.-butyl phenol, hydrochinone, and mixtures thereof, more preferably sodium parabens.

Preferred amides include, but are not limited to, diazolidinyl urea, 2,4-imidazolidinedione (HYDATOIN), 3,4,4′-trichlorocarbanilide, 3-trifluoromethyl-4,4′-dichlorocarbanilide, undecylenic acid monoethanolamide, and mixtures thereof, more preferably 2,4-imidazolidinedione.

Preferred acids include, but are not limited to, ascorbic acid, a benzoate, benzoic acid, citric acid, dehydroacetic acid, potassium sorbate, salicylic acid derivatives (acetyl salicylic acid and salicylic acid aldehyde), sodium citrate, sodium dehydroacetate, sodium salicylate, sorbic acid, undecylenic acid, zinc undecylenate, and mixtures thereof; more preferably benzoic acid, citric acid, salicylic acid, and sorbic acid; more preferably still citric acid and sorbic acid.

Preferred nitro compounds include, but are not limited to, 2-bromo-2-nitro-2,3-propanediol (BRONOPOL), methyldibromoglutaronitrile, and propylene glycol (MERGUARD), and mixtures thereof.

Further preferred antimicrobial agents are 2,5-dimethoxytetrahydrofurane, 2,5-diethoxytetrahydrofuran, 2,5-dimethoxy-2,5-dihydrofuran, 2,5-diethoxy-2,5-dihydrofuran, succindialdehyde, glutar-dialdehyde, glyoxal, glyoxylic acid, hexahydrotriazine, tetrahydro-3,5-dimethyl-2H-1,3,5-thiadiazine-2-thione (DAZOMET), 2,4-dichlorobenzyl alcohol, benzalkonium chloride, chlorhexidine gluconate, and triclosan.

Masking agents include coacervate perfume encapsulation by a commonly known encapsulation method. Small amounts of one or more perfume ingredients listed above are enclosed in a solid wall material. Water-soluble cellular matrix microcapsules are described in detail in U.S. Pat. No. 5,429,628, which is incorporated herein by reference. Water-soluble cellular matrix microcapsules are used for time-delay release of the perfume ingredient.

Addition of compounds of transition metals, such as Cu, Ag, and Zn.

Addition of enzymes (urease-inhibitors).

Further compounds for odor control are low pH-derivatives, peroxides, hydrogen carbonates, extracts of vegetables. etherical oils, boron derivatives, poly-alpha-aminoacids, imides, polyimides, pvp-iodine, chitosan, polyglycoside, and cyclophanes.

Odor control means in particle and/or powder form and/or aqueous solution comprising

optionally a solubilized uncomplexed cyclodextrin to absorb malodors;

optionally an antimicrobial agent to reduce growth of bacteria;

optionally a perfume ingredient, optionally with masking agents. Small amounts of one or more perfume ingredients listed above are enclosed in water-soluble cellular matrix microcapsules for timed release of the perfume ingredient;

optionally an enzyme to improve odor control benefit.

Preferably used odor control means are selected from the group consisting of zeolites, bentonite, silica, cyclodextrins, aminopolycarboxylic acids, perfumes, antimicrobial agents, and enzymes.

Another group of odor control means are polymers containing acidic groups and/or anhydride groups. These polymers differ from superabsorbent polymers in that they are not crosslinked. Their pH is below 6.5, preferably below 5.5, for example in the range of from pH 4.2 to pH 5.0.

Suitable monomers for the preparation of the uncrosslinked, acidic and/or anhydride groups containing polymers are those listed above under superabsorbent polymer-forming monomer. Preferred are polymerizable, unsaturated, acidic groups containing monomers. Examples of olefinically unsaturated carboxylic acid and carboxylic acid anhydride monomers are monoethylenically unsaturated C₃-C₂₅ carboxylic acids or anhydrides, such as acrylic acid, methacrylic acid, ethacrylic acid, crotonic acid, α-acryloxypropionic acid, sorbic acid, α-stearylacrylic acid, maleic acid, maleic acid anhydride, itaconic acid, citraconic acid, mesaconic acid, glutaconic acid, aconitic acid, fumaric acid, itaconic acid anhydride, and maleic acid anhydride.

Examples of olefinically unsaturated sulfonic acid and phosphonic acid monomers include aliphatic or aromatic vinyl sulfonic acids, such as vinylsulfonic acid, allylsulfonic acid, vinyltoluene sulfonic acid, and styrenesulfonic acid; acrylic and methacrylic sulfonic acids, such as sulfoethyl acrylate, sulfoethyl methacrylate, sulfopropyl acrylate, sulfopropyl methacrylate, styrene sulfonic acid, 2-hydroxy-3-acryloxypropyl sulfonic acid, 2-hydroxy-3-methacryloxypropyl sulfonic acid, and 2-acrylamido-2-methylpropane sulfonic acid; vinylphosphonic acid, allylphosphonic acid; and mixtures thereof.

Preferred monomers are acrylic acid, methacrylic acid, vinylsulfonic acid, 2-acrylamido-2-methylpropane sulfonic acid, and mixtures thereof, such as mixtures of acrylic acid and methacrylic acid, mixtures of acrylic acid and 2-acrylamido-2-methylpropane sulfonic acid, or mixtures of acrylic acid and vinylsulfonic acid.

Suitable odor controlling agents include copolymers of an acidic group containing monomer with one or more other monoethylenically unsaturated monomers. The above acidic monomers can be copolymerized, for example, with amides or nitrites of monoethylenically unsaturated carboxylic acids, such as acrylamide, methacrylamide, N-vinylformamide, N-vinylacetamide, N-methyl-vinylacetamide, acrylonitrile, and methacrylonitrile. Other comonomers are vinyl esters of saturated C₁-C₄ carboxylic acids, such as vinyl formates, vinyl acetate, vinyl propionate; alkyl vinyl ether compounds with at least two C-atoms within the alkyl group, such as ethyl vinyl ether and butyl vinyl ether, esters of monoethylenically unsaturated C₃-C₆ carboxylic acids, such as esters from primary C₁-C₁₈ alcohols and acrylic acid, methacrylic acid, or maleic acid; half esters of maleic acid, such as maleic acid monomethyl ester; N-vinyllactams, such as N-vinylpyrrolidone or N-vinylcaprolactam; acrylic acid or methacrylic acid esters of alkoxylated primary saturated alcohols, such as alcohols with 10 to 25 C-atoms which are reacted with 2 to 200 moles of ethylene oxide and/or propylene oxide per mole of alcohol; and monoacrylic acid esters and monomethacrylic acid esters of polyethylene glycol or polypropylene glycol up to a molecular weight of 2000. Further monomers are styrene and alkyl-substituted styrene compounds, such as ethylstyrene or tert-butylstyrene.

The copolymers may contain nonacidic monomers, for example, in an amount of from 0 to 60% by weight, preferably less than 20% by weight.

Preferred polymers used as odor control means are homopolymers of acrylic acid, homopolymers of methacrylic acid, copolymers of acrylic and methacrylic acid, copolymers of acrylic acid and maleic acid, and copolymers of methacrylic acid and maleic acid. These polymers may have an average molecular weight Mw of from 1,000 to 5 million, preferably from 1,500 to 150,000.

Examples of suitable odor controlling means are amphiphilic copolymers which contain units of

(a) hydrophobic monoethylenically unsaturated monomers, and

(b) monoethylenically unsaturated carboxylic acids, maleic anhydride, monoethylenically unsaturated sulfonic acids, monoethylenically unsaturated phosphonic acids, or mixtures thereof.

These amphiphilic copolymers are prepared, for example, by polymerizing in an aqueous medium in the presence of at least one initiator, at least one hydrophobic monoethylenically unsaturated monomer

(a) selected from the group consisting of styrene, methylstyrene, ethylstyrene, acrylonitrile, methacrylonitrile, C₂-C₁₈ olefins, esters of monoethylenically unsaturated C₃-C₅ carboxylic acids and monohydric alcohols, vinyl alkyl ethers, vinyl esters, or mixtures thereof. From this group of monomers, isobutene, diisobutene, styrene, and acrylic esters, such as ethyl acrylate, isopropyl acrylate, n-butyl acrylate, and sec-butyl acrylate, and at least one hydrophilic monomer.

(b) selected from the group consisting of acrylic acid, methacrylic acid, maleic acid, maleic anhydride, itaconic acid, vinyl sulfonic acid, 2-acrylamidomethylpropanesulfonic acid, acrylamido-propane-3-sulfonic acid, 3-sulfopropyl acrylate, 3-sulfopropyl methacrylate, styrenesulfonic acid, vinylphosphonic acid, or mixtures thereof in polyerized form.

If the amphiphilic copolymers are not sufficiently water-soluble in the form of the free acid, they are used in the form of water-soluble salts, for example, the corresponding alkali metal, alkaline earth metal, and ammonium salts are used. These salts are prepared, for example, by partial neutralization of the free acid groups of the amphiphilic copolymers with a base, for example, sodium hydroxide solution, potassium hydroxide solution, magnesium oxide, ammonia, or amines, such as triethanolamine, ethanolamine, morpholine, tri-thylamine, or butylamine, are used for the neutralzation. Preferably, the acid groups of the amphihilic copolymers are neutralized with sodium hydroxide or ammonia. The pH of the neutralized polymer solutions is for example less than 6.5, preferably less than 5.0. The average molecular weight Mw of the amphiphilic copolymers is, for example, from 1000 to 5,000,000, preferably from 1500 to 150,000.

Particularly preferred amphiphilic copolyers contain

(a) from 95 to 45% by weight of isobutene, diisobutene, styrene, or mixtures thereof, and

(b) from 5 to 55% by weight of acrylic acid, methacrylic acid, maleic acid, monoesters of maleic acid, or mixtures thereof, as polymerized units. These copolymers may additionally contain

(c) further monomers as polymerized units. The copolymers can, if required, contain units of monoesters of maleic acid as polymerized further monomers (c). Such copolymers are obtainable, for example, by copolymerizing copolymers of styrene, diisobutene, isobutene, or mixtures thereof with maleic anhydride in the absence of water and reacting the copolymers with alcohols after the polymerization, from 5 to 50 mol % of a monohydric alcohol being used per mole of anhydride groups in the copolymer. Suitable alcohols are, for example, methanol, ethanol, n-propanol, isopropanol, n-butanol, isobutanol, and tert-butanol. However, it is also possible to react the anhydride groups of the copolymers with polyhydric alcohols, such as glycol or glycerol. Here, however, the reaction is continued only until one OH group of the polyhydric alcohol reacts with the anhydride group. If the anhydride groups of the copolymers are not all reacted with alcohols, the anhydride groups not reacted with alcohols undergo ring opening as a result of the addition of water.

Other suitable odor controlling polymers may be obtained by grafting of synthetic or natural polymers with one or more of the above acidic monomers. Suitable backbones for grafting are, for example, starch, cellulose, and derivatives thereof, polysaccharides and oligosaccharides, polyvinyl alcohol, polyalkylene oxides (preferably polyethylene oxide and/or polypropylene oxide), polyamines, polyamides, hydrophilic polyesters, galactomannans, guar derivatives, and alginates. Grafted copolymers also include hydrolyzed starch-acrylonitrile graft copolymers, partially neutralized hydrolyzed starch-acrylonitrile graft copolymers, starch-acrylic acid graft copolymers, and partially neutralized starch-acrylic acid graft copolymers. Other suitable odor controlling agents are, for example, saponified vinyl acetate-acrylic ester copolymers, hydrolyzed acrylonitrile polymers, and hydrolyzed acrylamide copolymers. These polymers may be used either independently or in the form of a mixture with two or more other monomers.

The blend formed in step (a) contains at least one odor control means, for example, in an amount of from 0.01 to 30% by weight. In the alternative, it does not contain an odor control means which then is added to the coated fabric after polymerization. The effective concentration range varies widely depending on the mechanism of the odor control means, for example, antimicrobials are used in amounts of from 0.01 to 1% by weight, whereas the amounts of inorganic materials, pH-controlling materials and chelants are usually in the range of from 1 to 30% by weight.

Skin Care Agents

Skin care agents are known in the art. Examples are camomile extract, aloe vera, panthotenate, and esters.

Initiators

The blend formed in step (a) contains one or more initiators. Suitable initiators include the initiators and initiator combinations described above as being useful in the production of superabsorbent polymer particles. In addition, it may be desirable to use initiators designed to decompose when subjected to ultraviolet light and/or electron-beam (“e-beam”) irradiation. Preferred initiators include water-soluble azo compounds, such as 2,2′-azobis(2-(2-imidazole-2-yl))propane dihydrochloride and 2,2′-azobis(amidino)propane dihydrochloride, water soluble benzophenones, such as 4-benzoyl-N,N,N-trimethylbenzenemethanaminium chloride, 2-hydroxy-3-(4-benzoylphenoxy)-3-N,N,N-trimethyl-1-propanaminium chloride monohydrate, 2-hydroxy-3-(3,4-dimethyl-9-oxo-9H-thioaxanthon-2-yloxy-N,N,N-trimethyl-1-propanaminium chloride, 2-hydroxy-1-[4-(hydroxyethoxy)phenyl]-2-methyl-1-propanone, 2-hydroxy-2-methyl-1-phenyl-propan-1-one, and 4-benzo-yl-N,N-dimethyl-N-[2-(1-oxo-2-propenyl)oxy]ethylbenzenemethaminium chloride.

In general, the sprayable blend contains one or more initiators at a level sufficient to initiate polymerization of the superabsorbent-forming monomer in the blend. The blend contains one or more initiators at a level sufficient to result in the complete polymerization of the superabsorbent polymer forming monomer in the said blend, generally at a level within the range of from 0.01 to 5.0, most preferably at a level from 0.2 to 2.0, percent by weight of superabsorbent-forming monomer in the blend. When using a combination of initiators in the blend, such as redox package, it is possible to incorporate one of the initiators, such as the reducing agent, into the blend along with the other components of the blend and incorporate one or more additional initiators, such as an oxidizing agent, into the blend just before the sprayable blend exits, for example, the nozzle of a spray equipment which is used to spray the sprayable blend onto the fibrous web. A particularly preferred combination of initiators includes both an azo initiator and 2-hydroxy-1-[4-(hydroxyethoxy)phenyl]-2-methyl-1-propanone.

Water

In addition to the superabsorbent polymer particles, monomers forming superabsorbent polymers and one or more initiators, the blend contains water. Generally, the blend contains sufficient water to render the Brookfield viscosity (measured at 20° C., 20 rpm, spindle 02) of the blend of at least 20 mpas. For example, the viscosity is in the range of from 20 to 1,000 mpas, preferably 20 to 400 mPas. The level of water in the blend is generally in the range from 40 to 80 percent, more preferably from 50 to 60 percent, by weight of the blend. Blends having a viscosity of up to 400 mPas are preferred, because they can be easily sprayed onto both sides of the web by spaying through a nozzle.

Softening Agent

If desired, a softening agent may be added to the foregoing components of the blend formed in step (a). The amount of softening agent used in the blend may be from 0.2 to 20% by weight, preferably 5 to 15% by weight. Softening agents include such compounds as quaternary ammonium compounds (e.g., di(hydrogenated) tallow dimethylammonium chloride, di(hydrogenated) tallow dimethylammonium methyl sulfate), and/or polyhydroxy compounds selected from glycerol, polyglycerols, polyoxyethylene glycols, polyoxypropylene glycols, or mixtures thereof. If a softening agent is added to the blend, glycerol is preferably used as softening agent.

Crosslinker

It is generally desirable to include in the blend formed in step (a) one or more crosslinkers. It is particularly preferred to use one or more crosslinkers in this blend, for example thermal, redox and/or UV initiators. Suitable crosslinkers include those described above for the preparation of the superabsorbent polymer particles. Preferred crosslinkers include ethoxylated and propoxylated trimethylolpropane triacrylate derivatives such as SR-9035 and SR-492 available from Sartomer Co., Inc., Exton, Pa. When used, the crosslinkers are present in the blend formed in step (a), for example, in an amount of from 0.05 to 5.0, preferably 0.1 to 1% by weight, based on the weight of the monomers forming the superabsorbent polymer.

The Blend

In the process, a blend is formed in step (a) by combining monomer forming superabsorbent polymers, superabsorbent polymer particles, optional odor control means or mixtures thereof, water, optional softening agents or mixtures thereof, and initiator. The blends preferably contain a softening agent. The most preferred softening agent is glycerol. Although the order of combining these materials is not particularly important, for safety reasons it is preferred to add the initiator last. The amounts of the individual components of the blend are set forth above and are generally selected such that the Brookfield viscosity (measured at 20° C., 20 rpm, spindle 02) is at least 20 mpas. The blend formed in step (a) may have, for example, a viscosity of from 20 to 1,000 mPas, preferably from 20 to 400 mPas, more preferably from 30 to 150 mPas, most preferably from 40 to 100 mpas. Many factors will influence the viscosity of the blend, including the chemical nature and size of the superabsorbent polymer particles and odor control means, the extent of neutralization of the superabsorbent polymer particles, the extent of neutralization of the one or more superabsorbent-polymer-forming monomers, and the concentration of the superabsorbent polymer particles.

After the blend has been prepared, it is applied onto both sides of a fibrous web in step (b1). The blend can be printed onto a web and is preferably sprayed in step (b1) onto a fibrous web. As used herein, spraying is intended to include any suitable means for generating and delivering droplets of liquid. The spraying can be achieved by any conventional spray equipment. The equipment can be airless, air-assisted airless, or can utilize pressurized air. One or more inert gases, such as nitrogen, argon, or helium, may be substituted for some or all of the air to assist in removal of oxygen from the sprayable blend during the spraying process.

The loading level of the blend applied to the fibrous web is highly dependent on the application of the absorbent article. The blend is applied to the fibrous web, for example, in amounts of from 20 to 800 g/m², with reference to the solids including the monomers. Preferably from 40 to 300 g/m², and most preferably from 60 to 150 g/m², of the solids including the monomers of the blend are applied onto the web. For example, a blend having a solids content of 50% by weight is applied to a fibrous web in an amount of from 40 to 1,600 g/m². If spray equipment is used for the application of the blend, it should be adjusted to vary the droplet size of the spray taking into account such factors as the size of the superabsorbent polymer particles in the sprayable blend and the desired particle size of superabsorbent polymer particles on the final product.

Alternatively, after the blend has been prepared, it is applied onto a fibrous web in step (b2). The blend can be printed onto a web and is preferably sprayed in step (b2) onto a fibrous web. As used herein, spraying is intended to include any suitable means for generating and delivering droplets of liquid. The spraying can be achieved by any conventional spray equipment. The equipment can be airless, air-assisted airless, or can utilize pressurized air. One or more inert gases, such as nitrogen, argon, or helium, may be substituted for some or all of the air to assist in the removal of oxygen from the sprayable blend during the spraying process.

The loading level of the blend applied to the fibrous web is highly dependent on the application of the absorbent article. For example, a blend having a solids content of 46% by weight is applied to a fibrous web in an amount of from 20 to 1,500 g/m². If the absorbent article is used as an absorbent core of a hygiene article, the amount of the blend having a concentration of 46% by weight is, for example, of from 1,000 to 1,500 g/m². Blends having another solids content are used in such amounts that a corresponding solids content is achieved on the coated web. The blend is preferably applied by spraying to the fibrous web. For example, a sprayable blend having a viscosity of from 20 to 400 mPas (measured at 20° C., 20 rpm, spindle 02) and having a solids content of 46% by weight is sprayed onto the web in amounts up to 1,500, preferably from 20 to 800 g/m², and most preferably from 60 to 300 g/m². The spray equipment should be adjusted to vary the droplet size of the spray taking into account such factors as the size of the superabsorbent polymer particles in the sprayable blend and the desired particle size of superabsorbent polymer particles on the final product.

The blend can be applied homogeneously to a web on one or both sides, or also can be applied to it in a pattern which corresponds to a desired distribution. For example, the edges of the web may have a higher loading than the other parts of the web, or it may contain a homogeneous coating having several strips of higher loadings.

In order to apply different loadings of a sprayable blend on a fibrous web, there are, for example, several possibilities:

a) changing the spraying angle by electronic variation of the opening of the spray nozzle for different loadings in x/y direction;

b) changing the spraying angle by variation of the distance between spray nozzle and web for different loadings in x/y direction;

c) raising the amount of sprayable blend at certain regions of the web for different loadings in z direction. The continuous feed to the inlet tubes (spray nozzles) is changed at certain intervals by raising the flow velocity of the sprayable blend in such a way that the resulting loading of the web is increased;

d) stopping the web at certain intervals during the continuous process for different loadings in z direction. The constant web transport velocity is interrupted or slowed down at certain intervals.

The spray equipment should be adjusted to vary the droplet size of the spray taking into account such factors as the size of the superabsorbent polymer particles in the sprayable blend and the desired particle size of superabsorbent polymer particles in the final product.

For example, in regions of higher loading, a sprayable blend having a solids content of 46% by weight is sprayed onto a fibrous web in an amount of from 200 to 1,200 g/m², preferably from 250 to 800 g/m², most preferably from 300 to 500 g/m². These loadings are representative of the preparation of absorptive cores used in hygiene articles.

In a preferred embodiment of the invention the blend is applied to the fibrous web in such a way that the superabsorbent particles are uniformly distributed on the fibrous web.

The Web

Suitable fibrous webs for the present invention include those made using synthetic polymeric fibers. The synthetic polymeric fibers may be formed from any polymeric material capable of forming fibers which can be formed into a fibrous web. Suitable polymeric material from which the synthetic polymeric fibers may be formed include polyolefins, such as polyethylene, polypropylene, and the like; polyesters, such as polyethylene terephthalate and the like; polyamides, such as nylon 6, nylon 6,6, poly(iminocarboxylpentamethylene), and the like; acrylics, and modified cellulosic material, such as cellulose acetate and rayon, as well as mixtures and copolymers thereof.

The synthetic polymeric fibers may be formed by meltblowing, through a spunbond process, by extrusion and drawing, or other wet, dry, and melt spinning methods known to those skilled in the art. The synthetic polymeric fibers from which the web is formed may have a discrete length or may be substantially continuous. For example, if the synthetic polymeric fibers are formed by meltblowing, the fibers may be substantially continuous (few visible ends). If the fibers are formed by extrusion and drawing to produce a tow, the tow may be used as produced or cut into staple fibers having a length, for example of from about 25 millimeters to about 75 millimeters or short cut into length of from about 1 millimeter to about 25 millimeters. The synthetic polymeric fibers may suitably have a maximum cross-sectional dimension of from about 0.5 micrometer to about 50 micrometers, as determined by microscopic measurement using an optical microscope and a calibrated stage micrometer or by measurement from Scanning Electron photomicrographs.

The fibrous web may be formed directly through a spunbond or meltblown process, or by carding or air-laying staple or short cut fibers. Other methods of forming fibrous webs known to those skilled in the art may be suited for use in the present invention. The web may subsequently be bonded to enhance structural integrity. Methods of bonding fibrous webs are known to those skilled in the art and include thermal bonding, point bonding, powder bonding, ultrasonic bonding, chemical bonding, mechanical entanglement, and the like. The fibers may be homogenous fibers or may be a core/sheath or side-by-side fibers known in the art as bicomponent fibers.

The fibrous web may be formed from a single type of synthetic polymeric fiber or may contain synthetic polymeric fibers formed from different polymeric materials, having different fiber lengths or maximum cross-sectional dimensions. For example, the web may comprise a mixture of (1) bicomponent fibers having a polyethylene sheath and a polypropylene core which bicomponent fibers have a maximum cross-sectional dimension of about 20 micrometers and a length of about 38 millimeters and (2) polyester fibers (polyethylene terephthalate) having a maximum cross-sectional dimension of about 25 micrometers and a length of about 38 millimeters. Fibers (1) and (2) may be combined in a weight ratio of from 1:99 to 99:1. The fibers may be uniformly mixed or may be concentrated at opposite planar surfaces of the fibrous web.

The web suitably comprises from about 10 to 100 weight percent, preferably from about 20 to 100 weight percent, more preferably from about 25 to 100 weight percent, and most preferably from about 50 to 100 weight percent synthetic polymeric fibers. In addition to the synthetic polymeric fibers, the web may contain from about 90 to 0 weight percent of a nonsynthetic polymeric fiber, such as wood pulp fluff, cotton liners, cotton, and the like.

In one preferred embodiment, the web contains synthetic polymeric fibers which are formed from a polymeric having a high wet modulus. The importance of the modulus of a material is discussed in the book “Absorbency” edited by P. K. Chatterjee (Elsevier, N.Y., 1985). A polymeric material will be considered to have a high wet modulus when it has a wet modulus greater than about 80 percent of its dry modulus as determined by ASTM (American Society for Testing and Materials) test method D 2101-91 using modified gauge lengths. It is often desired to form the synthetic polymeric fibers of the web from a polymeric material having a high wet modulus because such material generally forms fibrous webs which possess a relatively high degree of wet resiliency. The wet resilience of a fibrous web is related to the pore structure (while under load) of the fibrous web. As will be discussed in greater detail below, it is often desired that the web have a relatively high degree of wet resilience.

The pore structure (while under load) of a fibrous structure formed from fibers of a polymeric material will, as discussed above, relate to the wet and/or dry modulus of the constituent fibers. Wet modulus of the constituent fibers should be considered for fibers that may likely be wetted during use. For the purpose of estimating the effect of load on the pore structure of a fibrous structure formed from fibers of a polymeric material, the tensile modulus of the fiber, which can be related to the flexural rigidity of the fiber as shown in the book “Physical Properties of Textile Fibers” by W. E. Morton and J. W. S. Hearl (The Textile Institute, London, 1975), can be used.

As a general rule, the polymeric materials from which the synthetic polymeric fibers of the web are formed will be inherently hydrophobic. As used herein, the term “hydrophobic” describes a material which has a contact angle of water-in-air of greater then 90 degrees. The term “hydrophilic” refers to a material which has a water-in-air contact angle of less than 90 degrees. The water-in-air contact angle is suitably determined as set forth in the book “Absorbency” edited by P. K. Chatterjee (Elsevier, N.Y., 1985). As used herein, a polymeric material will be considered as “inherently” hydrophobic or hydrophilic when the polymeric material, free from any surface modifications or treatments, e.g., surface active agent, spin fishes, blooming agents, etc., is hydrophobic or hydrophilic, respectively.

When the synthetic polymer fibers of the web are formed from a polymeric material which is inherently hydrophobic, it is often desirable to treat the fibers with a surface modifying material to render the surface of the fiber hydrophilic. For example, a surfactant may be applied to the fibers.

The web suitably has a basis weight of from about 20 to about 200 g/m², preferably from about 50 to about 150 g/m², and more preferably from about 25 to about 125 g/m².

The web suitably has a density of from about 0.005 to about 0.20 g/cm³, preferably from about 0.01 to about 0.16 g/cm³, and more preferably from about 0.08 to about 0.14 g/cm³.

The fibrous web may also comprise hydrophilic fibers. The hydrophilic materials may be inherently hydrophilic, such as cellulosic fibers, like wood pulp fluff, cotton linters, and the like; regenerated cellulose fibers, such as rayon; or certain nylon copolymers, such as poly(pentamethylenecarbonamide)(nylon-6)/polyethylene oxide. Alternatively, the hydrophilic fibers may be obtained from hydrophobic fibers by treatment with a hydrophilizing agent. For example, the fibers may be formed from a polyolefin material which is subsequently coated with a surface active agent such that the fiber itself is hydrophilic as described herein. Other methods of hydrophilizing fibers formed from hydrophobic materials are known and suited for use in the present invention.

Methods of providing inherently hydrophilic fibers, such as wood pulp fluff, are known. If the hydrophilic fibers are obtained from hydrophobic fibers which have been treated to possess a hydrophilic surface, the fibers will suitably have a fiber length and a maximum cross-sectional dimension as set forth above. If the hydrophilic fibers are inherently hydrophilic, such as wood pulp fluff, rayon, cotton, cotton linters, and the like, the fibers will generally have a length of from about 1.0 millimeters to about 50 millimeters and a maximum cross-sectional dimension of from about 0.5 micrometers to about 100 micrometers.

The fibrous web suitably comprises from about 10 to 100 weight percent, preferably from about 30 to 100 weight percent, and more preferably from about 55 to 100 weight percent of hydrophilic fibers, preferably inherently hydrophilic fibers. In addition to the hydrophilic fibers, the web may contain from about 90 to 0 weight percent of high wet modulus, preferably inherently hydrophobic fibers. The web may be formed from a single type of hydrophilic fiber or may contain hydrophilic fibers having different compositions, lengths, and maximum cross-sectional dimensions.

In one preferred embodiment, the web is formed from air-laid cellulosic fibers, such as wood pulp fluff. Wood pulp fluff fibers are preferred for use due to their ready availability and due to the fact that the fibers are relatively inexpensive compared to synthetic fibers.

In one especially preferred embodiment, the web is compressed under reduced pressure.

The compressed web suitably has a basis weight from about 40 to about 400 g/m², preferably from about 60 to about 300 g/m², and more preferably from about 50 to about 200 g/m².

The compressed web suitably has a density from about 0.15 to about 0.40 g/cm³, preferably from about 0.12 to about 0.30 g/cm³, and more preferably from about 0.10 to about 0.20 g/cm³.

It is a preferred embodiment of the present invention to provide profiled absorbent structures of at least two uniformly coated webs in which the characteristics of each individual web have been optimized to perform a specific function such as acquisition, fluid distribution, or fluid storage. An example of such an optimized structure would be a two layered structure in which one coated web has been optimized for fluid acquisition and the other for fluid storage. The performance characteristics of spray coated webs are dependent on a number of different factors, such as the x-link density and degree of loading of the formed superabsorbent polymer, as well as the density and basis weight of the substrate itself.

Spray coated webs optimized for fluid acquisition preferably are prepared from webs with a basis weight of 10 to 80 g/m², more preferably 20 to 60 g/m², with a density in the range of 0.005 to 0.08 g/cm³. The amount of applied superabsorbent polymer loading preferably is in the range of 10 to 80 percent by weight, more preferably 20 to 70% by weight, and most preferably 30 to less than 65% by weight.

It is further desired to use a highly crosslinked superabsorbent coating for acquisition layers to both minimize fluid retention and to increase swelling speed. Crosslinker amounts are preferably between 0.1 to 5.0%, more preferably between 0.3 to 4.0%, and most preferably between 0.5 to 3.0%, based on the amount of reactive monomer in the sprayable blend.

Spray coated webs optimized for fluid storage preferably are prepared from webs with a basis weight of 10 to 200 g/m², more preferably 40 to 150 g/m³, with a density in the range of 0.005 to 0.08 g/cm³. The amount of applied superabsorbent polymer loading preferably is in the range of 10 to 99 percent by weight, more preferably 20 to 90% by weight, and most preferably 30 to 85% by weight.

It is further desired to use a lightly crosslinked superabsorbent coating for storage layers to maximize absorptive capacity. Crosslinker amounts are preferably between 0.1 to 1.0%, based on the amount of reactive monomer in the sprayable blend.

Another desirable feature of the present invention is the incorporation of wicking layers in combination with webs coated with a sprayable blend to enhance fluid distribution within the described profiled absorbent structures. These distribution layers not only provide fluid transport in the x-y plane of the absorbent article, which promotes better utilization of the total available absorptive capacity of the absorbent article, but also provides drainage of the pore volume of the hydrated spray coated webs, which yields improved surface dryness as measured by rewet tests.

Curing/Polymerizing

After the blend has been applied onto the fibrous web, this composite is subjected to conditions under which the monomer forming the superabsorbent polymer will polymerize. Depending upon the type of initiator used in the blend, these conditions may include, for example, subjecting the fibrous web to which the blend was applied to heat, ultraviolet radiation, e-beam radiation, or a combination thereof. Furthermore, the composite can be subjected to static or continuous conditions, such as by moving the composite along a conveyor through regions of radiation or heat.

For thermal curing there are no particular limitations on the type of reaction vessel used. For batch polymerizations, sprayed webs may be cured in an oven in an air or inert atmosphere, and optionally under vacuum. In the case of a continuous process, the web may be passed through a dryer, such as an infrared (“IR”), through air or the like. The polymerization temperature can vary depending on the thickness of the substrate, the concentration of monomer, and the type and amount of thermal initiator used in the blend. The polymerization typically is carried out in the temperature range, for example, of from 20° C. to 150° C., and preferably from 40° C. to 100° C. The polymerization time depends on the polymerization temperature, but is typically several seconds to 2 hours, and preferably several seconds to 10 minutes. After polymerization is completed, the web then can be dried to the desired moisture content.

UV curing of webs coated with the sprayable blends may be conducted by the use of a conventional UV lamp. The conditions under which the irradiation is conducted, such as radiation intensity and time, may differ depending on the type of fibrous substrate used, the amount of monomer applied to the substrate, and the like. However, irradiation generally is conducted using a UV lamp with an intensity in the range of from 100 to 700 watts per inch (“W/in”), preferably in the range of from 400 to 600 W/in for 0.1 seconds to 10 minutes, with the distance between the UV lamp and the substrate being 2 to 30 centimeters. The irradiation of the composite with ultraviolet rays may be conducted under vacuum, in the presence of an inorganic gas, such as nitrogen, argon, helium, and the like, or in air.

The temperature during irradiation is not critical, and the irradiation of the sprayed web can be satisfactorily conducted at room temperature.

Electron beam curing can be accomplished using a commercially available electron beam accelerator, such as the ELECTROCURTAIN® CB 175 (Energy Sciences, Inc., Wilmington, Mass.). Accelerators operating in the 150 to 300 kilovolt range are acceptable. The beam current on such systems, typically 1 to 10 milliamperes, can be adjusted to obtain the desired dose of ionizing radiation. The ionizing radiation dose employed will vary somewhat, depending on factors such as the presence or absence of crosslinking monomers, desired degree of polymerization of the polymer, degree of cross-linking desired, and the like. In general, it is desirable to irradiate the coated web with doses from about 1 to 16 megarads, more preferably 2 to 8 megarads. Particularly when using lower doses, it is desirable to purge oxygen from the sprayable blend (as by bubbling nitrogen through the solution). The maximum dose would be that dose at which degradation of the fibers begins.

After irradiation, the coated web may be dried to remove water by such means as forced air ovens, infrared lamps, and the like.

Storage Layer

The absorbent structures according to the present invention are suitable for use in disposable absorbent products, such as diapers, training pants, adult incontinence products, feminine care products, wound dressings, and the like. Methods of forming such absorbent products and the absorbent products formed thereby are known to those skilled in the art and are described, for example, in the following U.S. Pat. Nos.: 4,944,735 issued Jul. 31, 1990 to Mokry; 4,798,603 issued Jan. 17, 1989, to Meyer et al.; 4,710,187 issued Dec. 1, 1987, to Boland et al.; 4,770,656 issued Sep. 13, 1988, to Proxmire et al.; and 4,762,521 issued Aug. 9, 1988, to Roessler et al., the disclosures of which are incorporated herein to the extent they are consistent herewith. The absorbent articles according to the invention can be used in disposable hygiene products as storage layer, wicking layer, and acquisition layer. They can be designed in such a way that they can substitute the absorbent core, the storage layer, and storage and acquisition layer in a conventional disposable hygiene article.

The absorbent structures of the present invention suitably form a storage layer or combined storage/acquisition layer (dual layer) of a disposable absorbent product. Such a storage or dual layer core is suitably sandwiched between, and in liquid communication with, a bodyside liner (also known as a top sheet), and a liquid impervious or relatively liquid inpervious outer cover. In order to function well as a storage layer or dual layer, an absorbent structure should exhibit rapid uptake of fluid, good transfer properties, good uptake upon repeated insults with fluid, good skin compatibility, low rewet, and high capacity on storage.

The absorbent structures of the present invention exhibit these properties and also have further advantages. Because the absorbent structures of the present invention can be prepared with superabsorbent polymer particles that are at least partially neutralized, and because the superabsorbent forming monomer used in the sprayable blend can be at least partially neutralized, it is possible to control the pH of the resulting absorbent structure formed by polymerizing the sprayable blend sprayed onto the fibrous web. By controlling the pH of the absorbent structure, particularly within the range of from about 4.3 to about 5.5, several advantages can result. For example, an absorbent structure having a pH within that range should be compatible with skin, should exhibit reduced bacterial growth, should reduce fecal proteolytic activity and lipolylic enzymatic activity, and should control odor and contain ammonia. The benefits associated with the control of pH in a top sheet of a disposable hygienic article are discussed, for example, in U.S. Pat. No. 4,657,537, the disclosure of which is hereby incorporated by reference. Similar benefits are expected to result from an acquisition layer having a pH in the range of from about 4.3 to about 5.5. Although the partial neutralization of the superabsorbent polymer particles and superabsorbent forming monomer will tend to reduce the overall absorptive capacity of the absorbent structure of the present invention, total absorptive capacity is not the only critical feature when using the structures as a dual layer in a disposable hygiene product.

It is further believed that the absorbent structures of the present invention perform well as storage and dual layers in a disposable diaper because the swelling of the superabsorbent polymer particles is capable of expanding the fibrous web, particularly if the web were compressed, so that the interstitial pore volume of the web increases after an insult of liquid. This increase in the interstitial pore volume of the web contributes to the rapid uptake of fluids in the web. Accordingly, enhancing the speed at which the interstitial pore volume is generated, such as by the rapid swelling of the superabsorbent polymer particles, further contributes to the rapid uptake of fluids in the web.

The free-swell expansion volume (“FSEV”) test and the expansion volume under load (“EVUL”) test described below are an indirect measure of the rate of formation of the interstitial pore volume in the web. In general, it is desirable to maximize the values of the FSEV and EVUL in designing a high performing superabsorbent article. At a minimum, the absorbent structures of the present invention have free-swell expansion volumes and/or expansion volumes under load of at least about 0.1 milliliter within about 5 minutes, preferably within about 30 seconds, and most preferably within about 5 seconds. Preferably, the absorbent structures of the present invention have free-swell expansion volumes of at least about 0.5 milliliter within about 5 minutes, preferably within about 30 seconds, and most preferably within about 5 seconds. Most preferably, the absorbent structures of the present invention generally have free-swell expansion volumes of at least about 1.0 milliliter within about 5 minutes, preferably within about 30 seconds, and most preferably within about 5 seconds. Preferably, the absorbent structures of the present invention generally have expansion volumes under load of at least about 0.2 milliliter within about 5 minutes, preferably within about 30 seconds, and most preferably within about 5 seconds. Most preferably, the absorbent structures of the present invention generally have expansion volumes under load of at least about 0.3 milliliter within about 5 minutes, preferably within about 30 seconds, and most preferably within about 5 seconds.

The dual layer may have an FSEV of at least about 0.1 ml within 5 minutes to about 1.0 ml within about 5 seconds, and an EVUL of at least about 0.1 ml within about 5 minutes to about 0.3 ml within about 5 seconds.

The absorbent core comprises a storage layer or a dual layer comprising a separate storage layer or a storage layer function. The storage layer or storage layer function is present in the absorbent structure in an amount from about 50 to about 100, preferably from about 60 to about 95, and most preferably from about 70 to about 90 weight percent, based on total weight of the absorbent core. If an additional acquisition layer is used, it is present in an amount of about 50 or less, preferably from 5 to 40, most preferably from 10 to 30 percent, based on the total weight of the absorbent core.

Means of containing acquisition layer are known to those skilled in the art. Any means of acquisition layer is suited for use in the present invention. The storage layer or dual layer may be combined with other high absorbency material.

As used herein, the term “high-absorbency material” refers to a water swellable, generally water insoluble, material capable of absorbing at least about 10, desirably about 20, and preferably about 50 times or more, its weight in water. The high-absorbency material may be formed from organic material, which may include natural materials such as agar, pectin, and guar gum, as well as synthetic materials, such as synthetic hydrogel polymers. Synthetic hydrogel polymers include, for example, carboxymethylcellulose, alkali metal salts of polyacrylic acid, polyacrylamides, polyvinyl alcohol, ethylene maleic anhydride copolymers, polyvinyl ethers, hydroxypropylcellulose, polyvinyl morpholinone, polymers and copolymers of vinylsulfonic acid, polyacrylates, polyacrylamides, polyvinyl-pyridine, polyvinylamines, and the like. Other suitable polymers include hydrolyzed acrylonitrile grafted starch, acrylic acid grafted starch, isobutylene maleic anhydride copolymers, and mixtures thereof. The hydrogel polymers preferably are lightly crosslinked to render the materials substantially water insoluble. Crosslinking may, for example, be by irradiation or covalent, ionic, van der Waals, or hydrogen bonding. Suitable materials are available from various commercial vendors such as the Dow Chemical Company, BASF Corporation, Nippon Shokubai., and Stockhausen Inc. The high-absorbency material may be in the form of particles, spheres, flakes, fibers, rods, films, or any of a number of geometric forms. When in the form of particles or spheres, it may be desired that the particles or spheres have a maximum cross-sectional dimension of from about 10 micrometers to about 2000 micrometers, and preferably from about 60 micrometers to about 1000 micrometers.

In one embodiment, it is desired that the high-absorbency material have the ability to absorb a liquid while under a load. The ability of a high-absorbency material to absorb a liquid while under a load is quantified as the Absorbency Against Pressure (AAP) value. Specifically, the AAP value is the amount (in grams) of an aqueous solution containing 0.9 weight percent sodium chloride that a gram of the high-absorbency material can absorb in 60 minutes under a load of 0.3 pound per square inch. As a general rule, it is desired that the high-absorbency material have an AAP value of at least about 10, desirably at least about 15, and preferably at least about 25.

The absorbent core suitably has a basis weight of from about 200 to about 1000 g/m², preferably from about 250 to about 750 g/m², and more preferably from about 300 to about 500 g/m². The absorbent layer suitably has a density from about 0.06 to about 0.40 g/cm³, preferably from about 0.12 to about 0.35 g/cm³, and more preferably from about 0.15 to about 0.30 g/cm³.

A further advantage of locating the dual layer between the body of a wearer and the absorbent layer is that the acquisition layer may have a relatively dry feel even after it has been wetted. This is because the acquisition layer includes synthetic polymeric fibers, is resilient, and may be more easily desorbed by the absorbent layer. Thus, a relatively dry surface may be presented for contacting a wearer's skin. In contrast, the absorbent layer including cellulosic or other inherently hydrophilic fibers can have a relatively wet feel. This wet surface is located remote from the body of a wearer and is spaced therefrom by the acquisition layer.

It is well known to those in the art of disposable hygienic products that the insertion of thick, lofty fabric structures between the topsheet and the absorbent core aids in the rate of uptake of fluid insults from the surface of the article. U.S. Pat. No. 5,364,382 discloses a number of key properties of these acquisition layers, such as the wet and/or dry modulus of the constituent fibers, the hydrophilicity of the fibers, and resiliency of the fabric structure, that contribute to the ability of these materials to rapidly uptake fluids in an absorbent article. Such properties contribute to the acquisition layer's ability to stay open under load, maintain void volume, resist collapse when wetted, enhance the desorption properties of the fabric, and preserve void volume capacity after successive insults of fluid.

One advantage of the absorbent articles of the present invention is the rate of formation of void volume following compression of the absorbent article. In many current, commercially manufactured absorbent products, a considerable amount of pressure is applied during manufacture to produce an “ultrathin” product. Acquisition and storage materials used in these current products have no real mechanism to reopen after being compressed other than the memory effects preserved within the fibers themselves. The incorporation of discrete superabsorbent particles into a fibrous web, as provided in the present invention, provides such a mechanism through swelling of the superabsorbent particles following an insult of fluid. Two important performance parameters of an acquisition and storage layer in a disposable hygienic article are (1) the degree of expansion and (2) the rate of expansion. Both of these parameters are indirectly measured by FSEV and EVUL. The degree of expansion is an indication of the pore volume available for fluid uptake (i.e., larger volume correlates with better performance), and by increasing the speed at which this pore volume is generated, the likelihood of leakage upon insult is diminished. Both of these properties may be influenced by adjusting such parameters as the degree loading of superabsorbent polymer particles on the web, the particle size of the superabsorbent polymer particles, the degree of swelling of the superabsorbent polymer particles, the degree of neutralization of the superabsorbent forming monomer in the sprayable blend, the degree of crosslinking, and the like.

It also has been observed that webs, after being sprayed with a sprayable blend and subjected polymerization conditions, have other beneficial properties. As mentioned above, certain web materials are subjected to compression at one or more times during the construction of a disposable hygienic article, such as a diaper. After a web material has been compressed, there is a tendency for the fibers to relax and expand somewhat, thereby increasing the thickness of the web. However, this relaxation phenomenon is much less pronounced in articles prepared in accordance with the present invention which tend to remain stably in a compact state until subjected to an insult of fluid.

Rewet and strikethrough testing of absorbent articles are common quality assurance tests used in the hygiene industry to measure surface dryness and the rate of fluid uptake, respectively, following successive fluid insults. Therefore, these tests are useful for evaluating the performance of acquisition materials in absorbent products. A common undesirable trend seen among most commercially available diapers on the market today is the fact that strikethrough times tend to increase with successive doses of fluid during rewet testing. With conventional fluff-based absorbent structures the cellulosic fibers can lose resiliency and collapse when wetted. As a result, the liquid uptake rate of the wetted structures may become too low to adequately accommodate subsequent, successive fluid insults. When absorbent gelling particles are incorporated between the fibers to hold them apart, the gelling particles swell and do not release the fluid. Swelling of the particles then can diminish the void volume of the absorbent structure and reduce the ability of the structure to rapidly uptake fluid. The degree to which the swelling of the absorbent gelling particle negatively impacts the rate of fluid uptake is dependent upon a number of factors, such as the concentration of superbsorbent used in the absorbent core, the degree of crosslinking, the uniformity of the distribution of SAP within the structure, the particle size distribution, the hydrophobicity of the particle, and the like. Each of these factors is easily controlled by the present invention and may be optimized to achieve the desired performance properties for a given absorbent article, particularly when used as an acquisition layer in an absorbent article.

In general, it can be seen that these acquisition and storage materials minimize or eliminate the trend of increasing strikethrough times with successive insults of fluid. This desired beneficial effect may be controlled and optimized by the present invention through the control of such parameters as the concentration of superabsorbent polymer particles applied to the web structure, the particle size distribution of the resulting superabsorbent polymer particles, the rate of swelling of the particles, the degree of swelling of the particles, and the like. A further observed beneficial effect is the reduction in rewet values. This effect also may be controlled through the above-described parameters. In addition, it is further believed that lowering the degree of neutralization of the superabsorbent particles formed on the web, thereby increasing the hydrophobicity of the particles, further enhances this effect by increasing it's tendency to be drained by the underlying wood fluff pulp/superabsorbent polymer absorbent core.

It is well known in the art that a hygienic absorbent article capable of lowering skin pH within the range of 3.0 to 5.5 is beneficial in preventing or at least reducing the incidence of diaper rash. Articles, compositions, and procedures which inherently tend to lower the pH of diapered skin are also known in the art. U.S. Pat. Nos. 4,657,537; 4,382,919; 3,964,486; 3,707,148; and 3,794,034 teach the addition of various acidic pH control agents to absorbent articles or to the diapered skin environment. In those instances wherein acidic pH control agents have been incorporated into the cores of the absorbent articles, significant amounts of acids are needed to bring about the desired absorption of ammonia or lowering of skin pH. Such approaches suffer from a number of drawbacks including: decreasing the absorptive capacity of the absorbent core, safety and comfort factors associated with leaching of the materials from the article, and processing problems associated with the placement and distribution of the acidic material within the absorbent core. U.S. Pat No. 4,657,537 discloses the preparation and use of topsheet materials containing ion exchange functionalities capable of controlling skin pH in urine soiled baby diapers. However, the ion-exchange capacities of these material are limited to the range of 0.25 to 1.0 meq/gram. Acquisition materials produced by the present invention may be prepared to contain from 1.0 to 10 meq/gram of ion-exchanging functionalities.

Test Methods

The centrifuge retention capacity (“CRC”) is a measure of the amount of fluid retained after being centrifuged. The CRC of the fabrics prepared in the Examples was determined as follows: a 5 centimeter diameter circle of the fabric was cut in half and one of the halves was placed into a teabag (6 cm×8.5 cm). The weight of the fabric prior to placing in the teabag was recorded. The teabag was sealed and placed in 0.9% saline solution for 20 minutes, then centrifuged for three minutes at 1350 revolutions per minute. The weight of the centrifuged teabag was measured and the CRC, in grams per square meter, was determined using the following formula:

CRC=(Wt.2−Wt.1−Blank)/A;

Wt.1=sample wt.;

Wt.2=teabag wt after centrifuged;

Blank=wt average of two measurements of an empty teabag after centrifuging;

A=area of sample in square meters [(πr²)/2] or 0.000982.

The free swell expansion volume (FSEV) is determined by measuring the height (thickness) change, in millimeters, of a compressed web material during hydration. The FSEV of the fabrics indicated in the Examples, were determined as follows: The fabrics were compressed in a Carver Laboratory Press Model #2697 at 7000 pounds of applied load for 48 seconds with the top platen heated to 50° C. A 5 centimeter diameter circle of the fabric was cut from the fabric and the thickness was measured before compression at approximately 4.5 millimeters, and after compression at approximately 0.67 millimeters, using a Fowler Ultra-digit gauge. The weight of the circle was recorded and the circle was placed in a dry sample holder. A single 30 milliliter dose of 0.9% saline was poured on top of the circle, and height measurements were taken, with the assistance of software designed for this purpose, over a 30-minute timeframe every 1.5 seconds. The change in the height of the fabric was measured with a linear variable differential transformer (LVDT, Schaevitz MP-1000) and the data are reported in milliliters (volume) at a chosen time interval.

The expansion volume under load (EVUL) is determined by measuring the volume change of a sample as height (thickness) change, in millimeters, of a compressed web material during hydration while under a load. The EVUL is determined in a similar manner as the FSEV except that a 55.93 gram weight (0.5 psi load) is applied to the fabric.

FSEV and EVUL values obtained between 5-30 seconds are useful for the characterization of webs designed for use as acquisition layers, whereas values obtained after 30 minutes are useful for the characterization of webs intended to be used as storage layers.

The viscosity was measured in a Brookfield viscometer at a temperature of 20° C. at 20 rpm using spindle 02.

### EXAMPLES

The term “superabsorbent polymer particles” used in the Examples means particles of a crosslinked polyacrylic acid having a degree of neutralization of 70% by mole, said particles were partially neutralized with sodium hydroxide and surface crosslinked. The following acquisition layers, also can be used alone as dual layers.

### Example 1

Layered absorbent structure containing an acquisition layer in combination with a storage layer

(a) Preparation of Acquisition Layer

An aqueous solution of acrylic acid was prepared by combining 306 grams of acrylic acid, 334 grams of deionized water, 250 grams of a 50% strength by weight aqueous sodium hydroxide solution, 9.1 grams of SR-9035 available from Sartomer (ethoxylated trimethylolpropane triacrylate), 0.75 grams of IRGACURE 2959 (4-(2-hydroxyethoxy)phenyl-(2-hydroxypropyl) ketone, CAS reg. No. 106797-53-9) from Ciba, 0.45 g of ammonium persulfate, and 0.75 grams of 2,2′-azobis[2-(2-imidazolin-2-yl)propane]-dihydrochloride. The aqueous solution of acrylic acid was purged with nitrogen. Superabsorbent polymer particles were sieved through a 100 mesh screen, and particles passing through having a particle sizes below 150 μm (the mean particle diameter was 80 μm) were collected. A sprayable blend was prepared by adding 23 grams of said superabsorbent polymer particles to the aqueous acrylic acid solution prepared above under stirring. The resulting sprayable mixture had a viscosity of 53 mPas. This mixture was sprayed using a commercial airless paint spraying apparatus onto both sides of a 0.056 m² piece of a commercially available polyester nonwoven fabric (HDK-210) having a basis weight of 55 g/m². The total amount of sprayable mixture retained by the fabric was 10.1 grams (wet load).

The fabric then was cured by ultraviolet radiation (“UV”) by passing the fabric under a 600 watt/in mercury vapor lamp, using a conveyor operating at 24 feet per minute. The fabric was passed under the lamp twice, turned over, and passed under the lamp another two times for a total exposure of 3.75 Joules of UVA radiation per cm² on each side. Following the UV cure, the fabric was dried in an oven for 10 minutes at 150° C. The weight of polymer in the resulting fabric was 82.9 g/m² (60% by weight of polymer and 40% by weight of polyester nonwoven fabric). The resulting web had a CRC of 7.8 g/g or 1,076 g/m². The FSEV and EVUL after 5 seconds for the web were 1.9 and 0.8 ml, respectively.

(b) Preparation of Storage Layer

An aqueous solution of acrylic acid was prepared by combining 306 grams of acrylic acid, 334 grams of deionized water, 250 grams of a 50% strength by weight aqueous sodium hydroxide solution, 1.8 grams of SR-344 available from Sartomer (polyethylene glycol diacrylate 400), 1.15 grams of DAROCUR 1173 (phenyl 2-hydroxy-2-propyl ketone) from Merck, 0.15 g of ammonium persulfate, and 0.75 grams of 2,2′-azobis[2-(2-imidazolin-2-yl)propane] dihydrochloride. The aqueous solution of acrylic acid was purged with nitrogen. Superabsorbent polymer particles were sieved through a 100 mesh screen, and the particles passing through having particle sizes below 150 μm (the mean particle diameter was 80 μm) were collected. A sprayable blend was prepared by adding 23 grams of these superabsorbent polymer partides with particle size below 150 μm to the aqueous acrylic acid solution prepared above under stirring. The resulting sprayable mixture had a viscosity of 56 mPas. This mixture was sprayed using a commercial airless paint spraying apparatus onto both sides of a 0.056 m² piece of a polyester nonwoven fabric (HP-15 available from Hobbs Industries) having a basis weight of 80 g/m². The total amount of sprayable mixture retained by the fabric was 55 grams (wet load).

The fabric then was cured by ultraviolet radiation (“UV”) and dried as described above. The weight of polymer in the resulting fabric was 453 g/m². The coated fabric consisted of 85% by weight of polymer and 15% by weight of polyester nonwoven fabric). The resulting coated web had a CRC of 23.0 g/g or 12,259 g/m². The FSEV and EVUL after 30 minutes for the web were 27 and 18 ml, respectively.

(c) Preparation of a Layered Absorbent Core Structure

The acquisition and storage layers prepared as described above were laid on top of each other and compressed in a Carver Laboratory Press Model #2697 at 7000 pounds of applied load for 48 seconds with the top platen heated to 50° C. The resulting compressed two layered absorbent composite structure had a thickness of 2.1 mm which was maintained until hydration. The profiled absorbent structure had a CRC of 12,988 g/m² and a FSEV and EVUL after 5 seconds of 3.8 and 2.0 ml, respectively. FSEV and EVUL values after 30 minutes were 34 and 19.5, respectively.

### Example 2

Layered absorbent structure containing a lowered pH acquisition layer to control skin pH in combination with a storage layer

(a) Preparation of Acquisition Layer

An aqueous solution of acrylic acid was prepared by combining 368.5 grams of acrylic acid, 441 grams of deionized water, 90.2 grams of 50% strength by weight aqueous sodium hydroxide solution, 9.1 grams of SR-9035 available from Sartomer (ethoxylated trimethylolpropane triacrylate), 0.75 grams of 4-(2-hydroxyethoxy)phenyl 2-hydroxypropyl ketone (IRGACURE 2959 from Ciba), 0.45 g of ammonium persulfate, and 0.75 grams of 2,2′-azobis[2-(2-imidazolin-2-yl)propane] dihydrochloride. The aqueous solution of acrylic acid was purged with nitrogen. Superabsorbent polymer particles (12 g), which were sieved through a 100 mesh screen, and had a particle size below 150 μm (mean particle diameter was 45 μm), were added under stirring to the aqueous acrylic acid solution prepared above. The resulting mixture was sprayable and had a viscosity of 63 mpas. This mixture was sprayed using a commercial airless paint spraying apparatus onto both sides of a 0.056 m² piece of a polyester nonwoven fabric (HDK-210) having a basis weight of 55 g/m². The total amount of sprayable mixture retained by the fabric was 17.1 grams (wet load).

The fabric then was cured by ultraviolet radiation (“UV”) by passing the fabric under a 600 watt/in mercury vapor lamp, using a conveyor operating at 24 feet per minute. The fabric was passed under the lamp twice, turned over, and passed under the lamp another two times for a total exposure of 3.75 Joules of UVA radiation per square centimeter on each side. Following the UV cure, the fabric was dried in an oven for 10 minutes at 150° C. The weight of polymer in the resulting coated fabric was 83.7 g/m² (the coated material consisted of 60% by weight of polymer and 40% by weight of fibers in the form of nonwoven fabric). The resulting web had a CRC of 4.0 g/g or 335 g/m². The FSEV and EVUL after 5 seconds for the web were 0.8 and 0.4 ml, respectively. The pH of the surface of the web was 4.5 (measured after the determination of CRC using a Ross Flat Surface Combination pH electrode, Model #8235).

(b) Preparation of Storage Layer

An aqueous solution of acrylic acid was prepared by combining 306 grams of acrylic acid, 334 grams of deionized water, 250 grams of 50% strength by weight aqueous sodium hydroxide solution, 1.8 grams of SR-344 available from Sartomer (polyethylene glycol diacrylate 400), 1.15 grams of phenyl 2-hydroxy-2-propyl ketone (DAROCUR 1173 from Merck), 0.15 g of ammonium persulfate, and 0.75 grams of 2,2′-azobis[2-(2-imidazolin-2-yl)propane] dihydrochloride. The aqueous solution of acrylic acid was purged with nitrogen. A sprayable blend was prepared by adding under stirring 23 grams of superabsorbent polymer particles having a particle size below 150 μm (obtained by sieving superabsorbent particles through a 100 mesh screen, the mean particle diameter was 45 μm) to the aqueous acrylic acid solution prepared above. The resulting sprayable mixture had a Brookfield viscosity of 56 mPas. This mixture was sprayed using a commercial airless paint spraying apparatus onto both sides of a 0.056 m² piece of a polyester nonwoven fabric (HP-15 available from Hobbs Industries) having a basis weight of 80 g/m². The total amount of sprayable mixture retained by the fabric was 55 grams (wet load). The fabric then was cured by ultraviolet radiation (“UV”) and dried as described above. The weight of polymer in the resulting coated fabric was 453 g/m². The coated nonwoven fabric consisted of 85% by weight of polymer and 15% by weight of fibers in the form of nonwoven fabric. The resulting web had a CRC of 23.0 g/g or 12,259 g/m². The FSEV and EVUL after 30 minutes for the web were 27 and 18 ml, respectively.

(c) Preparation of Layered Absorbent Core Structure

The acquisition and storage layers prepared as described above were laid on top of each other and compressed in a Carver Laboratory Press Model #2697 at 7000 pounds of applied load for 48 seconds with the top platen heated to 50° C. The resulting compressed two layered absorbent composite structure had a thickness of 2.3 mm which was maintained until hydration. The profiled absorbent structure had a CRC of 12,509 g/m² and a FSEV and EVUL after 5 seconds of 2.7 and 1.0 ml, respectively. FSEV and EVUL values after 30 minutes were 31 and 17.5, respectively.

### Example 3

Layered absorbent structure containing an acquisition layer in combination with a storage layer and wicking layers

(a) Preparation of the Acquisition Layer

A sprayable blend was prepared as described in Example 1(a). A polyester nonwoven substrate (HP-15) having a weight of 80 g/m² and consisting of two thermally bonded 40 g/m² layers was used in this Example. A 0.056 m² section of this nonwoven substrate was carefully separated with gentle tearing to produce two individual sheets with a basis weight of approximately 40 g/m². One of these sheets was coated on both sides with the sprayable mixture by spraying with a commercial airless paint spraying apparatus. The total amount of the sprayable mixture retained by the fabric was 16.5 grams (wet load). The fabric then was cured by ultraviolet radiation (“UV”) and dried as described in Example 1. The weight of polymer in the resulting fabric was 135 g/m² (the coated fabric consisted of 77% by weight of polymer and 23% by weight of polyester fibers). The resulting coated web had a CRC of 8.8 g/g or 1,540 grams per square meter. The FSEV and EVUL after 5 seconds for the web were 1.9 and 1.0 ml, respectively.

(b) Preparation of Storage Layer

A sprayable blend was prepared as described in Example 1(b) above and sprayed using a commercial airless paint spraying apparatus onto both sides of the other 0.056 m² half sheet described in Example 3(a), i.e., the sheet obtained by separating the two sheets of the polyester nonwoven sample HP-15. The total amount of sprayable mixture retained by the fabric was 16.5 grams (wet load). The fabric then was cured by ultraviolet radiation (“UV”) and dried as described in Example 1. The sheet coated on both sides consisted of 85% by weight of polymer and of 15% by weight of fibers. The weight of the coating was 227 g/m². The resulting web had a CRC of 23.2 g/g or 6194 grams per square meter. The FSEV and EVUL after 30 minutes for the web were 12.2 and 7.0 ml, respectively.

(c) Preparation of Layered Absorbent Core Structure With Wicking Layers

The acquisition layer of Example 3(a) and the storage layer of Example 3(b) were each individually placed in a laboratory pad former and a 125 g/m² layer of wood fluff pulp was air-laid onto a single side of each web. The acquisition and storage layers thus prepared were laid on top of each other with the fluff pulp containing side pointing downwards and compressed in a Carver Laboratory Press Model #2697 at 7000 pounds of applied load for 48 seconds with the top and bottom platens heated to 50° C. The resulting compressed four layered absorbent composite structure had a thickness of 3.1 mm which was maintained until hydration. The profiled absorbent structure had a CRC of 8,892 grams per square meter and a FSEV and EVUL after 5 seconds of 2.7 and 1.0 ml, respectively. FSEV and EVUL values after 30 minutes were 15.2 and 8.9 ml, respectively. Wicking and fluid distribution properties were greatly improved when compared to layered absorbent core structures not containing wood fluff layers.

### Examples 4 and 5

The following two commercial diapers were used to prepare the experimental samples described below:

Diaper A: medium, adult brief

Core: homogeneous wood fluff/superabsorbent polymer pad

Weight ratio of wood fluff to superabsorbent polymer in core: 38.8 grams: 7.2 grams

Secondary chemically treated mechanical pulp (“CTMP”) core, 23.0 grams

Random, pinpointed bonded top sheet

Density of core: 0.08 grams per cubic centimeter

Density of secondary CTMP core: 0.09 grams per cubic centimeter

Diaper B: medium, baby

Core: homogeneous wood fluff/superabsorbent polymer pad

Weight ratio of wood fluff to superabsorbent polymer in core: 19.7 grams: 10.3 grams

Polypropylene, point bonded top sheet

Density of core: 0.15 grams per cubic centimeter.

Experimental diapers were prepared by stretching out Diaper A and Diaper B and taping them to a laboratory bench top. The experimental fabric, as indicated in Table 1 below, was used to replace both the acquisition layer and wood fluff/superabsorbent polymer cores in each diaper. A razor knife was used to carefully cut along the edges of the top sheet to expose the diaper core. In the case of Diaper A, the secondary CTMP core along with the wood fluff/superabsorbent polymer core were removed and replaced with the compressed experimental fabric as indicated in Table 1 below. In the case of Diaper B, the high loft nonwoven (basis weight of 45 g/m²) and the wood fluff/superabsorbent polymer core were removed and replaced with the compressed experimental fabric as indicated in Table 1 below.

TABLE 1

| | Commercial Diaper | Fabric from |
|---|---|---|
| Example 4 | Diaper A | Example 3 |
| Example 5 | Diaper B | Example 3 |

The commercial and experimental diapers were evaluated with a rewet test, which measures the amount of unabsorbed liquid left on the surface of a wetted diaper. The rewet values reported in Table 2, were determined by taping the diaper to a Plexiglas□ board or to the counter top, and insulting the center of the diaper with 0.9% saline using a dosing ring (5 centimeter inside diameter with 2.5 centimeter height). Diaper A and the absorbent structure of Example 4 were insulted with 100 milliliters of the saline solution. Diaper B and the absorbent structure of Examples 5 were insulted with 80 milliliters of the saline solution. The amount of time needed for the liquid to be absorbed from the surface of the diaper was recorded as the strikethrough time. After 20 minutes, a stack of Whatman #3, 9.0 cm diameter filter paper (W1) was weighed and placed on top of the insulted area. A 3620 gram weight was placed on the filter paper for two minutes. The stack of filter paper was reweighed and the weight recorded (W2). This procedure was repeated two more times, recording the filter paper weight change and the strikethrough times.

The rewet values were calculated as follows:

W2−W1=rewet value (grams).

The rewet and strikethrough data reported in Table 2, below, are the average values of the results conducted on three separate diapers.

TABLE 2

| | Rewet (grams) | | | Strikethrough Time (seconds) | | |
|---|---|---|---|---|---|---|
| | 1st | 2nd | 3rd | 1st | 2nd | 3rd |
| Example | Dose | Dose | Dose | Dose | Dose | Dose |
| Commercial | 0.8 | 14 | 29 | 15.5 | 19.7 | 25.8 |
| Diaper A | | | | | | |
| Example 4 | 0.1 | 2.0 | 4.0 | 10.1 | 12.3 | 18.5 |
| Commercial | 0.08 | 0.15 | 8.0 | 7.5 | 7.6 | 16.3 |
| Diaper B | | | | | | |
| Example 5 | 0.00 | 0.03 | 2.0 | 4.9 | 5.2 | 12.1 |

### Example 6

Preparation of Acquisition Layer

Acidic Acquisition Layer, pH=4.5

An aqueous solution of acrylic acid was prepared by combining 368.5 grams of acrylic acid, 441 grams of deionized water, 90.2 grams of 50% w/w sodium hydroxide, 9.1 grams of SR-9035 available from Sartomer (ethoxylated trimethylolpropane triacrylate), 0.75 grams of 4-(2-hydroxyethoxy)phenyl 2-hydroxypropyl ketone (IRGACURE 2959 from Ciba), 0.45 g of ammonium persulfate, and 0.75 grams of 2,2′-azobis[2-(2-imidazolin-2-yl)propane] dihydrochloride. The aqueous solution of acrylic acid was purged with nitrogen. Superabsorbent polymer partides were sieved through a 100 mesh screen and the particles passing through (having particle sizes below 150 μm, the mean particle diameter was 80 μm) were collected. A sprayable blend was prepared by adding 12 grams of the collected superabsorbent polymer particles to the aqueous acrylic acid solution prepared above and stirring. The resulting sprayable mixture had a viscosity of 63 mPas. The sprayable mixture was sprayed using a commercial airless paint spraying apparatus onto both sides of a 0.056 square meter piece of a polyester nonwoven fabric (HDK-210) having a basis weight of 55 g/m². The total amount of sprayable mixture retained by the fabric was 17.1 grams (“wet load”). The fabric then was cured by ultraviolet radiation (“UV”) by passing the fabric under a 600 watt/in mercury vapor lamp, using a conveyor operating at 24 feet per minute. The fabric was passed under the lamp twice, turned over and passed under the lamp another two times for a total exposure of 3.75 Joules of UVA radiation per square centimeter on each side. Following the UV cure, the fabric was dried in an oven for 10 minutes at 150° C. The weight of polymer in the resulting fabric was 83.7 g/m² (60% by weight of polymer and 40% by weight of fibers). The resulting web had a CRC of 4.0 g/g or 335 grams per square meter. The FSEV and EVUL after 5 seconds for the web were 0.8 and 0.4 ml, respectively. The pH of the surface of the web was measured following the determination of CRC using a Ross Flat Surface Combination pH electrode (Model #8235). The pH was determined to be 4.5.

### Example 7

Layered absorbent structure containing a lowered pH and acquisition layer to control skin pH in combination with a storage layer

(a) Preparation of Acquisition Layer

An acquisition layer was prepared as described in Example 6.

(b) Preparation of Storage Layer

An aqueous solution of acrylic acid was prepared by combining 306 grams of acrylic acid, 334 grams of deionized water, 250 grams of 50% w/w sodium hydroxide, 1.8 grams of SR-344 available from Sartomer (polyethylene glycol diacrylate 400), 1.15 grams of phenyl 2-hydroxy-2-propyl ketone (DAROCUR 1173 from Merck), 0.15 g of ammonium persulfate, and 0.75 grams of 2,2′-azobis[2-(2-imidazolin-2-yl)propane] dihydrochloride. The aqueous solution of acrylic acid was purged with nitrogen. Superabsorbent polymer particles were sieved through a 100 mesh screen and the particles passing through (having particle sizes below 150 μm, the mean particle diameter was 80 μm) were collected. A sprayable blend was prepared by adding 23 grams of the collected superabsorbent polymer particles to the aqueous acrylic acid solution prepared above and stirring. The resulting sprayable mixture had a Brookfield viscosity of 56 cps. The sprayable mixture was sprayed using a commercial airless paint spraying apparatus onto both sides of a 0.056 square meter piece of a polyester nonwoven fabric (HP-15 available from Hobbs Industries) having a basis weight of 80 grams per square meter. The total amount of sprayable mixture retained by the fabric was 55 grams (“wet load”). The fabric was then cured by ultraviolet radiation (“UV”) and dried as described above. The weight of polymer in the resulting coated fabric was 453 g/m² (85% by weight polymer and 15% by weight fibers). The resulting web had a CRC of 23.0 g/g or 12,259 g/m². The FSEV and EVUL after 30 minutes for the web were 27 and 18 ml, respectively.

(c) Preparation of Layered Absorbent Core Structure

The acquisition and storage layers prepared as described above were laid atop one another and compressed in a Carver Laboratory Press Model #2697 at 7000 pounds of applied load for 48 seconds with the top platen heated to 50° C. The resulting compressed two layered absorbent composite structure had a thickness of 2.3 mm, which was maintained until hydration. The profiled absorbent structure had a CRC of 12,509 grams per square meter and a FSEV and EVUL after 5 seconds of 2.7 and 1.0 ml, respectively. FSEV and EVUL values after 30 minutes were 31 and 17.5, respectively.

### Examples 8-9

The following two commercial diapers were used to prepare the experimental samples described below:

Diaper A: medium, adult brief

Core: homogeneous wood fluff/superabsorbent polymer pad

Weight ratio of wood fluff to superabsorbent polymer in core: 38.8 grams: 7.2 grams

Secondary chemically treated mechanical pulp (“CTMP”) core, 23.0 grams

Random, pinpointed bonded top sheet

Density of core: 0.08 grams per cubic centimeter

Density of secondary CTMP core: 0.09 grams per cubic centimeter

Diaper B: medium, baby

Core: homogeneous wood fluff/superabsorbent polymer pad

Weight ratio of wood fluff to superabsorbent polymer in core: 19.7 grams: 10.3 grams

Polypropylene, point bonded top sheet

Density of core: 0.15 grams per cubic centimeter

Experimental diapers were prepared by stretching out Diaper A and Diaper B and taping them to a laboratory bench top. The experimental fabric, as indicated in Table 3 below, was compressed under a 7000 pound load for 48 seconds using a Carver laboratory press with the top platen heated to 50° C. The thickness of the fabric following compression was 0.67 millimeters. An Exacto razor knife was used to carefully cut along the edges of the top sheet to expose the diaper core. In the case of Diaper A, the secondary CTMP core was removed and replaced with the compressed experimental fabric as indicated in Table 3. In the case of Diaper B, the high loft nonwoven (basis weight of 45 grams per square meter) was removed and replaced with the compressed experimental fabric as indicated in Table 3.

TABLE 3

| | Commercial Diaper | Fabric from |
|---|---|---|
| Example 8 | Diaper A | Example 6 |
| Example 9 | Diaper B | Example 6 |

The commercial and experimental diapers were evaluated with a rewet test which measures the amount of unabsorbed liquid left on the surface of a wetted diaper. The rewet values reported in Table 4 were determined by taping the diaper to a Plexiglas® board or to the counter top, insulting the center of the diaper with 0.9% saline solution using a dosing ring (5 centimeter inside diameter with 2.5 centimeter height). Diaper A and Example 8 were insulted with 100 milliliters of the saline. Diaper B and Example 9 were insulted with 80 milliliters of the saline. The amount of time needed for the liquid to be absorbed from the surface of the diaper was recorded as the strikethrough time. After 20 minutes, a stack of Whatman #3, 9.0 cm diameter filter paper (W1) was weighed and placed on top of the insulted area. A 3620 gram weight was placed on the filter paper for two minutes. The stack of filter paper was reweighed and the weight recorded (W2). This procedure was repeated two more times, recording the filter paper weight change and the strikethrough times. The pH of the top sheet, the transfer layer (if any), and the core were measured after the experiment was complete using a Ross Flat Surface Epoxy Combination pH Model #8235 electrode. The data are reported in Table 5. The rewet values were calculated as follows:

W2−W1=rewet value (grams).

The rewet and strikethrough data reported in Table 4 are the average values of the results conducted on three separate diapers.

TABLE 4

| | | | | Rewet (grams) | | |
|---|---|---|---|---|---|---|
| | Rewet (grams) | | | Strikethrough Time (seconds) | | |
| | 1st | 2nd | 3rd | 1st | 2nd | 3rd |
| Example | Dose | Dose | Dose | Dose | Dose | Dose |
| Commercial | 0.8 | 14 | 29 | 15.5 | 19.7 | 25.8 |
| Diaper A | | | | | | |
| Example 8 | 1.1 | 8.1 | 18.4 | 4.4 | 4.8 | 4.9 |
| Commercial | 0.08 | 0.15 | 8.0 | 7.6 | 7.6 | 16.3 |
| Diaper B | | | | | | |
| Example 9 | 0.56 | 0.65 | 0.78 | 3.3 | 4.0 | 4.3 |

TABLE 5
pH Measurements of Diaper Surfaces

| | | Acquisition | |
|---|---|---|---|
| Example | Topsheet | Layer | Fluff/SAP Core |
| Commercial | 5.3 | 5.4 | 5.8 |
| Diaper A | | | |
| Example 8 | 4.7 | 4.4 | 5.1 |
| Commercial | 5.3 | 5.4 | 5.8 |
| Diaper B | | | |
| Example 9 | 4.5 | 4.5 | 5.1 |

### Example 10

Double-sided coated profiled absorbent structure containing an acquisition layer in combination with preparation of a storage layer

(a) Preparation of Storage Layer

An aqueous solution of acrylic acid was prepared by combining 306 grams of acrylic acid, 334 grams of deionized water, 250 grams of 50% w/w sodium hydroxide, 1.8 grams of SR-344 available from Sartomer (polyethylene glycol diacrylate 400), 1.15 grams of phenyl 2-hydroxy-2-propyl ketone (DAROCUR 1173 from Merck), 0.15 g of ammonium persulfate and 0.75 grams of 2,2′-azobis[2-(2-imidazolin-2-yl)propane] dihydrochloride. The aqueous solution of acrylic acid was purged with nitrogen. Superabsorbent polymer particles were sieved through a 100 mesh screen and the particles passing through (having particle sizes below 150 μm, the mean particle diameter was 45 μm) were collected. A sprayable blend was prepared by adding 45 grams of the collected superabsorbent polymer particles to the aqueous acrylic acid solution prepared above and stirring. The resulting sprayable mixture had a Brookfield viscosity of 56 mpas. The sprayable mixture was sprayed using a commercial airless paint spraying apparatus onto one side of a 0.056 square meter piece of a polyester nonwoven fabric (HP-15 available from Hobbs Industries) having a basis weight of 80 g/m². The total amount of sprayable mixture retained by the fabric was 55 grams (“wet load”). The fabric then was cured by ultraviolet radiation (“UV”) by passing the fabric under a 600 watt/in mercury vapor lamp, using a conveyor operating at 24 feet per minute. The fabric was passed under the lamp twice, turned over and passed under the lamp another two times for a total exposure of 3.75 Joules of UVA radiation per square centimeter on each side. Following the UV cure, the fabric was dried in an oven for 10 minutes at 150° C. The weight of polymer in the resulting fabric was 453 grams per square meter (85% by weight of polymer and 15% by weight of fibers). The resulting web had a CRC of 23.0 g/g or 12,259 g/m². The FSEV and EVUL after 30 minutes for the web were 27 and 18 ml, respectively.

(b) Preparation of Acquisition Layer

An aqueous solution of acrylic acid was prepared by combining 306 grams of acrylic acid, 334 grams of deionized water, 250 grams of 50% w/w sodium hydroxide, 9.1 grams of SR-9035 available from Sartomer (ethoxylated trimethylolpropane triacrylate), 0.75 grams of 4-(2-hydroxyethoxy)phenyl 2-hydroxypropyl ketone (IRGACURE 2959 from Ciba), 0.45 g of ammonium persulfate and 0.75 grams of 2,2′-azobis[2-(2-imidazolin-2-yl)propane] dihydrochloride. The aqueous solution of acrylic acid was purged with nitrogen. Superabsorbent polymer particles were sieved through a 100 mesh screen and the particles passing through (having particle sizes below 150 mm, the mean particle diameter was 45 μm) were collected. A sprayable blend was prepared by adding 22.5 grams of the collected superabsorbent polymer particles to the aqueous acrylic acid solution prepared above and stirring. The resulting sprayable mixture had a Brookfield viscosity of 63 mPas. The sprayable mixture was sprayed using a commercial airless paint spraying apparatus onto the uncoated side of the storage layer prepared as described above The total amount of sprayable mixture retained by the fabric was 17.1 grams (“wet load”). The fabric then was cured by ultraviolet radiation (“UV”) and dried as described above. The weight of additional superabsorbent polymer added to the resulting fabric was 83.7 grams per square meter (60% by weight of polymer and 40% by weight of fibers). The profiled absorbent structure had a CRC of 12,988 grams per square meter and a FSEV and EVUL after 5 seconds of 3.8 and 2.0 ml, respectively. FSEV and EVUL values after 30 minutes were 34 and 19.5, respectively.

### Example 11

Double-sided coated profiled absorbent structure containing a lowered pH acquisition layer in combination with a storage layer

(a) Preparation of Storage Layer

An aqueous solution of acrylic acid was prepared by combining 306 grams of acrylic acid, 334 grams of deionized water, 250 grams of 50% w/w sodium hydroxide, 1.8 grams of SR-344 available from Sartomer (polyethylene glycol diacrylate 400), 1.15 grams of phenyl 2-hydroxy-2-propyl ketone (DAROCUR 1173 from Merck), 0.15 g of ammonium persulfate, and 0.75 grams of 2,2′-azobis[2-(2-imidazolin-2-yl)propane] dihydrochloride. The aqueous solution of acrylic acid was purged with nitrogen. Superabsorbent polymer particles were sieved through a 100 mesh screen and the particles passing through (having particle sizes below 150 μm, the mean particle diameter was 80 μm) were collected. A sprayable blend was prepared by adding 45 grams of the collected superabsorbent polymer particles to the aqueous acrylic acid solution prepared above and stirring. The resulting sprayable mixture had a Brookfield viscosity of 56 cps. The sprayable mixture was sprayed using a commercial airless paint spraying apparatus onto one side of a 0.056 square meter piece of a polyester nonwoven fabric (HP-15 available from Hobbs Industries) having a basis weight of 80 g/m². The total amount of sprayable mixture retained by the fabric was 55 grams (“wet load”). The fabric then was cured by ultraviolet radiation (“UV”) by passing the fabric under a 600 watt/in mercury vapor lamp, using a conveyor operating at 24 feet per minute. The fabric was passed under the lamp twice, turned over and passed under the lamp another two times for a total exposure of 3.75 Joules of UVA radiation per square centimeter on each side. Following the UV cure, the fabric was dried in an oven for 10 minutes at 150° C. The weight of polymer in the resulting fabric was 453 g/m² (85% by weight of polymer and 15% by weight of fibers). The resulting web had a CRC of 23.0 g/g or 12,259 g/m². The FSEV and EVUL after 30 minutes for the web were 27 and 18 ml, respectively.

(b) Preparation of Acquisition Layer

An aqueous solution of acrylic acid was prepared by combining 368.5 grams of acrylic acid, 441 grams of deionized water, 90.2 grams of 50% w/w sodium hydroxide, 9.1 grams of SR-9035 available from Sartomer (ethoxylated trimethylolpropane triacrylate), 0.75 grams of 4-(2-hydroxyethoxy)phenyl 2-hydroxypropyl ketone (IRGACURE 2959 from Ciba), 0.45 g of ammonium persulfate, and 0.75 grams of 2,2′-azobis[2-(2-imidazolin-2-yl)propane] dihydrochloride. The aqueous solution of acrylic acid was purged with nitrogen. Superabsorbent polymer particles were sieved through a 100 mesh screen, and the particles passing through (having particle sizes below 150 μm, the mean particle diameter was 80 μm) were collected. A sprayable blend was prepared by adding 22.5 grams of the collected superabsorbent polymer particles to the aqueous acrylic acid solution prepared above and stirring. The resulting sprayable mixture had a Brookfield viscosity of 63 mPas. The sprayable mixture was sprayed using a commercial airless paint spraying apparatus onto the uncoated side of the web prepared as described above The total amount of sprayable mixture retained by the fabric was 17.1 grams (“wet load”). The fabric was then cured by ultraviolet radiation (“UV”) and dried as described above. The weight of additional superabsorbent polymer added to the resulting fabric was 83.7 grams per square meter (60% by weight of polymer and 40% by weight of fibers). The profiled absorbent structure had a CRC of 12,509 g/m² and a FSEV and EVUL after 5 seconds of 2.7 and 1.0 ml, respectively. FSEV and EVUL values after 30 minutes were 31 and 17.5, respectively. The pH of the surface of the web was measured following the determination of CRC using a Ross Flat Surface Combination pH electrode (Model #8235). The pH of the acquisition side of the was determined to be 4.5. The pH of the storage layer side of the web was 5.9.

### Example 12

Commercial adult brief incorporating an acquisition/storage layer core supplement.

The following commercial diaper was used to prepare the experimental sample described below:

Diaper A: medium, adult brief

Core: homogeneous wood fluff/superabsorbent polymer pad

Weight ratio of wood fluff to superabsorbent polymer in core: 38.8 grams: 7.2 grams

Secondary chemically treated mechanical pulp (“CTMP”) core, 23.0 grams

Random, pinpointed bonded top sheet

Density of core: 0.08 grams per cubic centimeter

Density of secondary CTMP core: 0.09 grams per cubic centimeter

Experimental diapers were prepared by stretching out Diaper A and taping them to a laboratory bench top. The experimental fabric, as prepared in Example 11, was compressed under a 7000 pound load for 48 seconds using a Carver laboratory press with both platens heated to 50° C. The thickness of the fabric following compression was 1.35 millimeters. An Exacto razor knife was used to carefully cut along the edges of the top sheet to expose the diaper core. The secondary CTMP core was removed and replaced with the compressed experimental fabric.

The commercial and experimental diapers were evaluated with a rewet test which measures the amount of unabsorbed liquid left on the surface of a wetted diaper. The rewet values reported in Table 6 below, were determined by taping the diaper to a Plexiglas□ board or to the counter top, insulting the center of the diaper with 0.9% saline solution using a dosing ring (5 centimeter inside diameter with 2.5 centimeter height). Diaper A and the absorbent member of Example 3 were insulted with 100 milliliters of the saline. The amount of time needed for the liquid to be absorbed from the surface of the diaper was recorded as the strikethrough time. After 20 minutes a stack of Whatman #3, 9.0 cm diameter filter paper (W1) was weighed and placed on top of the insulted area. A 3620 gram weight was placed on the filter paper for two minutes. The stack of filter paper was reweighed and the weight recorded (W2). This procedure was repeated two more times, recording the filter paper weight change and the strikethrough times. The pH of the top sheet, the transfer layer (if any),and the core were measured after the experiment was complete using a Ross Flat Surface Epoxy Combination pH Model #8235 electrode. The data are reported in Table 7, below:

The rewet values were calculated as follows:

W2−W1=rewet value (grams)

The rewet and strikethrough data reported in Table 6, below, are the average values of the results conducted on three separate diapers.

TABLE 6

| | Rewet (grams) | | | Strikethrough Time (seconds) | | |
|---|---|---|---|---|---|---|
| | 1st | 2nd | 3rd | 1st | 2nd | 3rd |
| Example | Dose | Dose | Dose | Dose | Dose | Dose |
| Commercial | 0.8 | 14 | 29 | 15.5 | 19.7 | 25.8 |
| Diaper A | | | | | | |
| Absorbent | 0.1 | 8.4 | 18.2 | 8.6 | 11.2 | 18.7 |
| member of | | | | | | |
| Example 12 | | | | | | |

TABLE 7
pH Measurements of Diaper Surfaces

| | | Acquisition | |
|---|---|---|---|
| Example | Topsheet | Layer | Fluff/SAP Core |
| Commercial | 5.3 | 5.4 | 5.8 |
| Diaper A | | | |
| Absorbent | 4.9 | 5.0 | 5.5 |
| member of | | | |
| Example 12 | | | |

Other layers which can be used as acquisition or dual layers are described in the following examples.

### Example 13

Acquisition Layer Containing an Odor Control Agent

An aqueous solution of acrylic acid was prepared by combining 306 grams of acrylic acid, 334 grams of deionized water, 250 grams of 50% w/w sodium hydroxide, 9.1 grams of SR-9035 available from Sartomer (ethoxylated trimethylolpropane triacrylate), 0.75 grams of 4-(2-hydroxyethoxy)phenyl 2-hydroxy-2-propyl ketone (IRGACURE 2959 from Ciba),0.45 g of ammonium persulfate, and 0.75 grams of 2,2′-azobis[2-(2-imidazolin-2-yl)propane] dihydrochloride. To the monomer mixture technical grade beta-cyclodextrin BW7 (available from Wacker Biochemicals) (15.3 g) was added as an odor control agent. The aqueous solution of acrylic acid was purged with nitrogen. Superabsorbent polymer particles were sieved through a 100 mesh screen, and the particles passing through (having particle sizes below 150 microns, mean particle diameter was 80 μm) were collected. A sprayable blend was prepared by adding 23 grams of the collected superabsorbent polymer particles to the aqueous acrylic acid solution prepared above and stirring. The resulting sprayable mixture had a Brookfield viscosity of 53 mPas. The sprayable mixture was sprayed using a commercial airless paint spraying apparatus onto both sides of a 0.056 m² piece of a polyester nonwoven fabric (HDK-210) having a basis weight of 55 grams per square meter. The total amount of sprayable mixture retained by the fabric was 10.1 grams (wet load). The fabric then was cured by ultraviolet radiation (“UV”) by passing the fabric under a 600 watt/in mercury vapor lamp, using a conveyor operating at 24 feet per minute. The fabric was passed under the lamp twice, turned over and passed under the lamp another two times for a total exposure of 3.75 Joules of UVA radiation per square centimeter on each side. Following the UV cure, the fabric was dried in an oven for 10 minutes at 150° C. The weight of polymer in the resulting fabric was 82.9 g/m² (60 wt %). The resulting web had a CRC of 7.8 g/g or 1.076 g/m². The FSEV and EVUL after 5 seconds for the web were 1.9 and 0.8 ml, respectively.

### Example 14

An acquisition layer was prepared identical to that in Example 13, except that 54.0 g of Bioshield AM 5001 (available from Bioshield Technologies, Inc., Norcross Ga.) was substituted for beta-cyclodextrin. The active ingredient in Bioshield AM 500 I is 3-(trimethoxysilyl)propyldimethyloctadecyl ammonium chloride.

### Example 15

An acquisition layer was prepared identical to that in Example 13, except that no odor control agent was added to the sprayable blend prior to polymerization. A solution was prepared by dissolving 0.10 g of IRGAGUARD B 1000 (triclosan, available from Ciba) in 100 ml of a 50/50 (v/v) propylene glycol/water solution. The solution was placed in a thin layer chromatography atomizing sprayer, and 3.0 g of the solution was applied to each side of the web. The coated web then was dried in an oven for 10 minutes at 150° C.

### Example 16

A 0.056 M² piece of a polyester nonwoven fabric (HDK-210) having a basis weight of 55 g/m² was coated with the IRGAGUARD B 1000 solution as described in Example 15. The resulting triclosan treated web then was used to prepare an acquisition layer as described in Example 13.

### Example 17

Layered absorbent structure containing an acquisition layer in combination with a storage layer incorporating odor control agents

(a) Preparation of Acquisition Layer

An acquisition layer was prepared as described in Example 14.

(b) Preparation of Storage Layer

An aqueous solution of acrylic acid was prepared by combining 306 grams of acrylic acid, 334 grams of deionized water, 250 grams of 50% w/w sodium hydroxide, 1.8 grams of SR-344 available from Sartomer (polyethylene glycol diacrylate 400), 1.15 grams of phenyl 2-hydroxy-2-propyl ketone (DAROCUR 1173 from Merck), 0.15 g of ammonium persulfate, and 0.75 grams of 2,2′-azobis[2-(2-imidazolin-2-yl)propane] dihydrochloride. To the monomer mixture 54.0 g of Bioshield AM 500 1 (available from Bioshield Technologies, Inc., Norcross, Ga.) was added as an odor control agent. The aqueous solution of acrylic acid was purged with nitrogen. Superabsorbent polymer particles were sieved through a 100 mesh screen, and the particles passing through (having particle sizes below 150 microns, the mean particle diameter was 45 μm) were collected. A sprayable blend was prepared by adding 23 grams of the collected superabsorbent polymer particles to the aqueous acrylic acid solution prepared above and stirring. The resulting sprayable mixture had a Brookfield viscosity of 56 mpas. The sprayable mixture was sprayed using a commercial airless paint spraying apparatus onto both sides of a 0.056 square meter piece of a polyester nonwoven fabric (HP-15 available from Hobbs Industries) having a basis weight of 80 g/m². The total amount of sprayable mixture retained by the fabric was 55 grams (“wet load”). The fabric then was cured by ultraviolet radiation (“UV”) and dried as described above. The weight of polymer in the resulting fabric was 453 grams per square meter (85 wt %). The resulting web hat a CRC of 23.0 g/g or 12.259 grams per square meter. The FSEV and EVUL after 30 minutes for the web were 27 and 18 ml, respectively.

(c) Preparation of Layered Absorbent Core Structure

The acquisition and storage layers prepared as described above were laid a top one another and compressed in a Carver Laboratory Press Model #2697 at 7000 pounds of applied load for 48 seconds with the top platen heated to 50° C. The resulting compressed two layered absorbent composite structure had a thickness of 2.1 mm which was maintained until hydration. The profiled absorbent structure hat a CRC of 12,988 grams per square meter, and a FSEV and EVUL after 5 seconds of 3.8 and 2.0 ml, respectively. FSEV and EVUL values after 30 minutes were 34 and 19.5, respectively.

What is claimed is:

1. A method of preparing a storage layer for aqueous fluids comprising
(a) forming a sprayable blend comprising one or more superabsorbent forming monomers, superabsorbent polymer particles, water, and one or more initiators;
(b) applying said sprayable blend to a fibrous web; and
(c) subjecting said sprayed fibrous web to conditions under which the superabsorbent forming monomers polymerize to form a storage layer for aqueous fluids.

2. The method of claim 1 wherein the storage layer has a CRC equal or greater 9,000 g/m².

3. The method of claim 1 wherein the storage layer has a CRC equal or greater 10,000 g/m².

4. The method of claim 1 wherein the storage layer has a CRC equal or greater 12,000 g/m².

5. The method of claim 1 wherein the storage layer further comprises an odor control agent, an agent having a skin care effect, or a mixture thereof.

6. The method of claim 1 wherein the sprayable blend has a viscosity at least 20 mPas.

7. The method of claim 1 wherein the sprayable blend formed in step (a) further comprises a crosslinking agent.

8. The method of claim 1 wherein the sprayable blend formed in step (a) further comprises a softening agent.

9. The method of claim 1 wherein the sprayable blend formed in step (a) contains at least one odor control agent, an agent having a skin care effect, or a mixture thereof.

10. The method of claim 1 wherein the superabsorbent polymer particles of the sprayable blend formed in step (a) contain at least one odor control agent, an agent having a skin care effect, or a mixture thereof.

11. The method of claim 1 wherein the storage layer has a pH of from 2.0 to 7.5.

12. The method of claim 1 wherein the storage layer has a pH of from 4.0 to 6.5.

13. The method of claim 1 wherein the superabsorbent particles comprise a mixed bed ion exchange superabsorbent polymers or multidomain composites.

14. A storage layer prepared by the method of claim 1.

15. An absorbent article comprising a storage layer prepared by the method of claim 1.

16. The absorbent article of claim 15 wherein the storage layer is a component of a double-sided coated fibrous web, wherein the opposite side of the fibrous web is an acquisition layer.
